(19) United States
(12) Patent Application Publication
(10) Pub. No.: US 2024/0159749 A1
(43) Pub. Date: May 16, 2024

(54) METHOD FOR DIAGNOSING PRIMARY BILIARY CIRRHOSIS (PBC) USING NOVEL AUTOANTIGENS

(71) Applicants: AMBERGEN, INC., Watertown, MA (US); THE GENERAL HOSPITAL CORPORATION, Boston, MA (US)

(72) Inventors: MARK J. LIM, Reading, MA (US); HEATHER P. OSTENDORFF, Framingham, MA (US); KENNETH J. ROTHSCHILD, Newton, MA (US); DONALD B. BLOCH, Newton, MA (US)

(73) Assignees: AMBERGEN, INC., Watertown, MA (US); THE GENERAL HOSPITAL CORPORATION, Boston, MA (US)

(21) Appl. No.: 18/522,114

(22) Filed: Nov. 28, 2023

Related U.S. Application Data
(62) Division of application No. 15/469,350, filed on Mar. 24, 2017, now Pat. No. 11,885,802.
Application No. 15/469,350 is a division of application No. 14/318,498, filed on Jun. 27, 2014, now abandoned.
Application No. 14/318,498 is a continuation of application No. 13/500,411, filed on Jun. 8, 2012, now Pat. No. 8,852,956.
(60) Provisional application No. 61/248,768, filed on Oct. 5, 2009.

(51) Int. Cl. G01N 33/564; G01N 33/68

(52) U.S. Cl. CPC G01N 33/564; G01N 33/6893; G01N 2333/47; G01N 2800/085

(57) ABSTRACT

Methods and compositions are described for the diagnosis of primary biliary cirrhosis. Novel autoantigens are described for use in assays which employ test samples from individuals.

### CROSS REFERENCE TO RELATED APPLICATIONS

This application is a divisional of U.S. application Ser. No. 15/469,350, filed Mar. 24, 2017, allowed, which is a divisional of U.S. application Ser. No. 14/318,498, filed Jun. 27, 2014, abandoned, which is a continuation of U.S. application Ser. No. 13/500,411, filed Jun. 8, 2012, now U.S. Pat. No. 8,852,956, which is a 371 of PCT/US2010/051475, filed Oct. 5, 2010, which claims benefit of U.S. Provisional Application No. 61/248,768, filed on Oct. 5, 2009, each of which is incorporated herein by reference in its entirety.

### GOVERNMENT LICENSE RIGHTS

This invention was made with government support under R44 A1052525 awarded by the National Institutes of Health. The government has certain rights in this invention.

### DESCRIPTION OF THE INVENTION

### Field of the Invention

This invention relates to molecular biology, biochemistry, cell biology, medicine and medical diagnostics. Specifically, the invention relates to novel nucleic acid molecules, proteins and polypeptide fragments encoded thereby, polyclonal and monoclonal antibodies thereto, and methods of using the nucleic acid molecules, proteins/polypeptides and antibodies in diagnostic, prognostic, staging and therapeutic regimens for the control of autoimmune disorders, viral diseases and cancers.

### Background of the Invention

More than 80 illnesses have been described that are associated with activation of auto-reactive lymphocytes and the production of autoantibodies directed against normal tissue or cellular components (autoantigens) [von Muhlen and Tan (1995) Semin Arthritis Rheum 24: 323-58; Mellors (2002) 2005]. Collectively referred to as autoimmune diseases, they are estimated to afflict 14.7-23.5 million people, up to 8% of the total U.S. population and constitute a major economic and health burden [Jacobson, Gange, Rose and Graham (1997) Clin Immunol Immunopathol 84: 223-43]. For unknown reasons, the number of people afflicted by autoimmune diseases is on the rise. An autoimmune diagnosis means a lifetime of illness and treatment, possible organ damage, debilitation and an increased chance of mortality. The chronic and often debilitating nature of autoimmune diseases results in poor patient health, increased medical costs, and decreased productivity. The root causes of the immune dysfunction underpinning autoimmune disease are still not well understood. Consequently, autoimmune diseases generally remain difficult to diagnose, due to the wide variability of clinical presentation, which typically involves a constellation of symptoms.

Autoimmune diseases are disorders in which an individual's immune system targets and destroys apparently normal tissue. Examples of autoimmune diseases include rheumatoid arthritis (RA), systemic lupus erythematosus (SLE), scleroderma (SCL), Sjogren's syndrome (SjS), polymyositis (PM), dermatomyositis (DM), mixed connective tissue disease (MCTD), pemphigus vulgaris (PV) and primary biliary cirrhosis (PBC). Autoantibodies are commonly directed against cellular proteins and nucleic acids. In certain diseases, such as PV, the target of autoantibodies is known and the autoantibody is thought to play a role in the pathogenesis of the disease. In other diseases, such as SLE, the targets of many different autoantibodies have been identified but the role of autoantibodies in the pathogenesis of SLE is as yet uncertain.

Detection of autoantibodies in the serum of patients assists in the diagnosis of autoimmune diseases. Rheumatoid factor (IgM antibodies directed against human IgG) is detected in the majority of patients with RA and supports that diagnosis in a given individual [Kelly, W. N., et al. 1985. Textbook of Rheumatology. 2nd ed. Saunders. pp. 667]. Antinuclear antibodies (ANA) are present in approximately 98% of individuals with active SLE. Although ANA are not specific for the diagnosis of SLE, the absence of these antibodies argues against the diagnosis of SLE in a given patient [Kelly et al., 1985 supra pp. 691].

Liver and biliary diseases collectively rank in the top ten causes of mortality in the U.S. Chronic liver diseases affect between 5 and 10 percent of Americans and cause 1 to 2 percent of deaths in the United States. Chronic liver disease and cirrhosis cost an estimated $1.6 billion per year [(2004)]. General causes of liver and biliary diseases include infectious agents, inherited defects, metabolic disturbances, alcohol, toxins and environmental toxicants. The most common liver diseases are chronic hepatitis C, alcohol liver disease, nonalcoholic fatty liver disease, chronic hepatitis B, autoimmune liver diseases and drug-induced liver diseases. Many of these conditions can be prevented or treated, but if not, they can lead to progressive liver injury, liver fibrosis and ultimately cirrhosis, portal hypertension, end-stage liver disease and, in some instances, liver cancer. Currently, the only therapy for end-stage liver disease is liver transplantation. More than 5,000 liver transplants are done in the U.S. each year. At least 17,000 persons are on a waiting list for liver transplantation and as many as 1,500 die yearly while waiting [(2004)]. Liver disease research presents many challenging needs. Autoimmune liver diseases include primary biliary cirrhosis (PBC), autoimmune hepatitis and primary sclerosing cholangitis. These chronic liver diseases can all lead to end-stage liver disease. Collectively, autoimmune liver diseases are responsible for 13% of adult liver transplants per year in the U.S. [(2004)].

PBC is a progressive cholestatic liver disease, with an estimated prevalence in the U.S. of approximately 40 adults per 100,000 population (incidence 2.7 per 100,000 U.S. population) [Kim, Lindor et al. (2000) Gastroenterology 119: 1631-6; Feld and Heathcote (2003) J Gastroenterol Hepatol 18: 1118-28; 2004)]. Women between the ages of 40 and 65 are predominantly affected by PBC, with a female to male ratio of 9:1 [Kaplan and Gershwin (2005) N Engl J Med 353: 1261-73], as is typical for autoimmune disease. PBC is characterized by the gradual progressive destruction of intrahepatic biliary ductules leading to hepatic fibrosis and liver failure (reviewed in [Kaplan (1996) N Engl J Med 335: 1570-80; Heathcote (2000) Hepatology 31: 1005-13; Kaplan (2002) Gastroenterology 123: 1392-4; Talwalkar and Lindor (2003) Lancet 362: 53-61]). PBC is a significant indication for liver transplantation, and PBC patients constitute 11% of all patients undergoing liver transplantation for cirrhosis [Milkiewicz (2008) Clin Liver Dis 12: 461-72; xi].

Treatment of PBC is accomplished with ursodeoxycholic acid (ursodiol), a natural bile acid that is not toxic to the liver, to replace the bile acids which are reduced by PBC. While the mechanisms are not fully understood, this treatment ultimately reduces intracellular build up of other liver-toxic bile acids (which was caused by bile duct destruction). Although ursodiol slows progression to cirrhosis, ursodiol treatment functions best when implemented early in the course of PBC, highlighting the importance of a rapid, reliable PBC diagnostic test. In fact, a study showed that ursodiol treatment at stages III and IV did not result in significant slowing of liver progression while patients treated early at histological stages I and II did show significant slowing of liver destruction with ursodiol treatment. This highlights the need for an early PBC diagnostic, to allow prompt medical treatment [Heathcote (2000) Hepatology 31: 1 005-13; Poupon, Lindor, Pares, Chazouilleres, Poupon and Heathcote (2003) J Hepatol 39: 12-6].

Roughly half of PBC patients first present with an abnormal blood test which triggers the eventual PBC diagnosis. Generally, diagnostic testing is initially activated by abnormal liver function tests and signs of bile disease, followed by testing for serum anti-mitochondrial autoantibodies (AMA), for which an estimated 87-95% of PBC patients test positive [Heathcote (2000) Hepatology 31: 1 005-13; Yang, Yu, Nakajima, Neuberg, Lindor and Bloch (2004) Clin Gastroenterol Hepatol 2: 1116-22; Kaplan and Gershwin (2005) N Engl J Med 353: 1261-73; Liu, Shi, Zhang, Zhang and Gao (2008) Liver Int 28:233-9]. Bile duct imaging tests are used to rule out other causes of biliary tract disease, and liver biopsies confirm diagnosis and provide a gauge of disease stage (based upon the degree of fibrosis).

However, the other roughly half of PBC patients will present only with a variety of relatively non-specific physical symptoms, highlighting the difficulties facing the general practitioner or specialist responsible for diagnosis. The most common of such symptoms are pruritis, fatigue and musculoskeletal pain [Prince, Chetwynd, Newman, Metcalf and James (2002) Gastroenterology 123: 1044-51 ]. Furthermore, numerous autoimmune disorders may be found in association with PBC, including autoimmune hepatitis(AIH) [Czaja (2006) J Hepatol 44: 251-2], thyroid dysfunction, sicca symptoms, Raynaud's syndrome, systemic lupus erythematosus (SLE) and rheumatoid arthritis [Heathcote (2000) Hepatology 31: 1005-13; Gershwin, Selmi, Worman, Gold, Watnik, Utts Lindor, Kaplan and Vierling (2005) Hepatology 42: 1194-202]. In one study, 19% of PBC patients were found to have features of another disease [Czaja (1998) Hepatology 28: 360-5], thereby clouding diagnosis. Of concern, the proper testing may not be ordered in many patients due to unrecognized etiology, especially when patients present with vague symptoms of pruritis or joint discomfort.

Autoantibodies have the potential to serve not only as diagnostic tools, but also as harbingers of the future development of PBC. In fact, anti-mitochondrial autoantibodies (AMA) have been shown to pre-date clinical manifestations and diagnosis of PBC [Metcalf, Mitchison, Palmer, Jones, Bassendine and James (1996) Lancet 348: 1399-402]. This demonstrates that it may be possible to diagnose PBC at an earlier stage using autoantibody biomarkers. The serological hallmark of PBC are AMA, which can be detected in 87-95% of patients [Kaplan (1996) N Engl J Med 335: 1570-80; Nishio, Keeffe and Gershwin (2002) Semin Liver Dis 22: 291-3021.] The major autoantigens targeted by these AMA include the E2 subunits of the pyruvate dehydrogenase complex (PDC-E2); the branched/chain 2-oxo-acid dehydrogenase complex (BCOADC-E2) and the the 2-oxo-glutarate dehydrogenase complex (OGDC-E2) [Fussey, Guest, James, Bassendine and Yeaman (1988) Proc Natl Acad Sci U S A 85: 8654-8; Nishio, Keeffe et al. (2002) Semin Liver Dis 22: 291-302].

Anti-nuclear autoantibodies (ANA) are present in ˜50% of PBC patients. Autoantibodies recognizing proteins of the nuclear core complex and multiple nuclear dots (MND) are useful PBC markers in AMA-negative patients, with a prevalence of 13-44% [Manuel Lucena, Montes Cano, Luis Caro, Respaldiza, Alvarez, Sanchez-Roman, Nunez-Roldan and Wichmann (2007) Ann N Y Acad Sci 1109: 203-11]. Additionally, ANA can serve as prognostic indicators, with anti-centromere and/or anti-nuclear pore glycoprotein 210 (gp210) autoantibodies being associated with liver failure in PBC [Yang, Yu et al. (2004) Clin Gastroenterol Hepatol 2: 1116-22; Nakamura, Kondo et al. (2007) Hepatology 45: 118-27].

The nuclear body(NB, also known as nuclear domain 10, PML oncogenic domain, and Kr body) is a nuclear organelle whose function is unknown [Ascoli, C. A., and Maul, G. G., J. Cell. Biol. 112:785-795 (1991); Brasch, K., and Ochs, R. L, Exp. Cell Res. 202:211-223 (1992); Dyck, J. A. et al., Cell 76:333-343 (1994)]. Using immunohistochemical staining, NBs appear as 5 to 30 discrete, punctate, dot-like regions within the nucleus. The NB is distinct from other nuclear domains including those involved in DNA replication and mRNA processing. In addition, components of the NB do not co-localize with kinetochores or centromeres [Brasch, K., and Ochs, R. L., Exp. Cell Res. 202:211-223 (1992)]. The number of NBs in the cell, and the intensity of antibody staining of these structures, increase in response to stimuli including interferons (IFNs), heat shock and viral infection [Ascoli, C. A., and Maul, G. G., J. Cell. Biol. 112:785-795 (1991)].

The NB is a target of autoantibodies in the serum of patients with the autoimmune disease primary biliary cirrhosis (PBC). Approximately 40% of patients with PBC have antibodies directed against this structure [Evans, J., et al., Arthr. Rheum. 347:31-736 (1991); Szostecki, C. et al., Scand. J. Immunol. 36:555-564 (1992)). Serum from patients with PBC was used to identify and characterize a 100-kDa component of the NB which was designated Sp100 (Speckled, 100 kDa) [Szostecki, C. et al., J. Immunol. 145:4338-4347 (1990)]. The fusion of Sp100 to the LexA DNA binding domain has been shown to activate gene transcription in Saccharomyces cerevisiae, and it has been suggested that Sp100 may participate in activation of transcription of specific regions in the genome [Xie, K. et al., Mol. Cell. Biol. 13:6170-6179 (1993)].

A second component of the NB, designated NDP52, was characterized using a murine monoclonal antibody that reacted with the NB [Korioth, F., et al., J. Cell Biol. 130:1-13 (1995)]. A cDNA encoding NDP52 was identified and the predicted amino acid sequence contained coiled coil, leucine zipper and zinc finger motifs. One or more of these domains may be involved in interactions between NDP52 and other components of the NB [Korioth, F., et al., J. Cell. Biol;. 130:1-13 (1995)].

A third component of the NB, PML, was identified by several investigators studying the t(15; 17) translocation associated with human acute promyelocytic leukemia (APL) [de The, H. et al., Nature (London) 347:558-561 (1990); Borrow, J. et al., Science 249:1577-1580 (1990); Longo, L. et al., J. Exp. Med. 172:1571-1575 (1990); Kakizuka, A. et al., Cell 66:663-674 (1991)]. In this translocation, the amino terminal portion of PML is fused to retinoic acid receptor alpha. PML was found to co-localize with Sp100 in the NB [Weis, K. et al., Cell 76:345-356 (1994); Koken, M. H. M. et al., EMBO 13:1073-1083 (1994)]. Expression of the PML-alpha fusion protein in APL cells appears to disrupt the NB; in these cells, the NB antigens are detected in numerous smaller regions in the nucleus described as “microspeckles.” Treatment of APL cells with retinoic acid (RA) results in differentiation of myeloid precursor cells and reformation of NBs [Dyck, J. A. et al., Cell 76:333-343 (1994); Weis, K. et al., Cell 76:345-356 (1994); Koken, M. H. M. et al., EMBO 13:1073-1083 (1994)]. In patients with APL, treatment with RA results in differentiation of leukemic cells and temporary disease remission [Warrell, R. P. et al., N. Eng. J. Med. 329:177-189 (1993)].

It is important to note however, that ANA are also found in a variety of other prevalent autoimmune disorders and a wide range of cancers [Bei, Masuelli, Palumbo, Modesti and Modesti (2008) Cancer Lett].

Indirect immunofluorescence (IIF) and solid-phase immunoassay are the two formats used to establish the presence or absence of autoantibodies in patients. Both methods have their pros and cons as discussed below:

For the past several decades, indirect immunofluorescence (IIF) has been the method of choice by physicians for the detection of autoantibodies present in the serum of autoimmune patients. Importantly, it remains the gold standard for AMA and ANA testing, including for PBC. Typically, patient serum is serial diluted in two-fold increments and allowed to bind to a cell substrate on a microscope slide (e.g. HEp-2 liver cells), which is then fluorescently stained to detect bound autoantibodies and examined under the microscope by a trained technician to identify the cellular/tissue staining patterns. IIF does have the advantage that as a cell/tissue based substrate, it can in theory “universally” cover all cellular autoantigens (pending their expression and preservation in the substrate). This, in part, is evidenced by the high diagnostic sensitivity of the IIF test, e.g. 93% (ANA) for systemic lupus erythematosus (SLE) [Solomon, Kavanaugh and Schur (2002) Arthritis Rheum 47: 434-44] and 90% (AMA) for PBC [Tanaka, Miyakawa, Luketic, Kaplan, Storch and Gershwin (2002) Cell Mol Biol (Noisy-le-grand) 48: 295-9].

Although IIF based AMA is a sensitive marker for PBC, the tradeoff may be specificity. Asymptomatic patients have been deemed AMA positive, and while a large portion only develop symptoms years later, some never develop symptoms at all [Metcalf, Mitchison et al. (1996) Lancet 348: 1399-402]. Moreover, one study found that 34% of AIH patients tested positive for AMA [Nezu, Tanaka, Yasui, Imamura, Nakajima, Ishida and Takahashi (2006) J Gastroenterol Hepatol 21: 1448-54].

Furthermore, the IIF assay is problematic overall when used as a routine diagnostic screening tool, as it is difficult to standardize owing to variations in the substrate and fixation process, variations in the microscopy apparatus. and due to the highly subjective interpretation of results [Jaskowski, Schroder, Martins, Mouritsen, Litwin and Hill (1996) Am J Clin Pathol 105: 468-73]. The consensus statement in 2004 from the Committee for Autoimmune Serology of the International Autoimmune Hepatitis Group (IAIHG) recommended that IIF be performed on three different organs from rodents [Vergani, Alvarez, Bianchi, Cancado, Mackay, Manns, Nishioka and Penner (2004) J Hepatol 41: 677-83). Both AMA and anti-liver kidney microsomal-1 (LKM1) antibodies stain the renal tubules of the kidney, with differences only apparent to the trained eye, and this confusion can lead to a diagnosis of autoimmune hepatitis (AIH) instead of PBC [Bogdanos, Invemizzi, Mackay and Vergani (2008) World J Gastroenterol 14: 3374-87]. Moreover, some autoantigens are lost (unrecognizable) by diffusion or denaturation during the fixation process of IIF. Another confounding factor is that multiple autoimmune diseases can often occur together in the same patient, and the overlapping IIF patterns can lead to confusion in the correct diagnosis of each [Assassi, Fritzler et al. (2009) J Rheumatol; Norman, Bialek, Encabo, Butkiewicz, Wiechowska-Kozlowska, Brzoska, Shums and Milkiewicz (2009) Dig Liver Dis 41: 762-4]. Finally, IIF is slow, laborious and not amenable to high-throughput automation [Ulvestad, Kanestrom, Madland, Thomassen, Raga and Vollset (2000) Scand J Immunol 52: 309-15].

Although IIF remains the gold standard in AMA testing, solid-phase immunoassays, such as ELISA (Enzyme Linked Immunosorbent Assay), are gaining popularity, especially in high-throughput laboratories [Fritzler and Fritzler (2006) Curr Med Chern 13: 2503-12]. These methods have the advantage of high throughput automation, high analytical sensitivity, purely objective scoring, reliability, and the ability to test for specific autoantigen species, including in a multiplexed fashion [Fritzler and Fritzler (2006) Curr Med Chern 13: 2503-12]. With a resolution at the individual antigen level, these methods have the potential for greater disease specificity, if the correct marker panel is chosen. The drawback, however, is that a sufficient number of autoantigens needs to be both discovered and clinically validated to match the diagnostic sensitivity of the cellular substrate based IIF assay.

In one example of a commercial solid-phase immunoassay for PBC, INOVA Diagnostics Inc. (San Diego, CA) markets the MIT3 assay, an FDA-approved ELISA-based immunoassay for PBC based on the detection of AMAs. The MIT3 is utilizes a recombinant protein containing the immunodominant epitopes of all three E2 subunits of the pyruvate dehydrogenase complex [Moteki, Leung, Cappel, Dickson, Kaplan, Munoz and Gershwin (1996) Hepatology 24: 97-103]. The overall goal of these tests is to mimic the cellular IIF-based AMA test for PBC, but with all the aforementioned benefits of solid-phase immunoassays of individual antigens. Still, this test is only meant to be diagnostic aid, together with clinicopathological findings for PBC. In one study, the AMA-based MIT3 ELISA assay had a reported a diagnostic sensitivity of 81.6%, however, it is important to note that serum samples with AMA-negative PBC disease were excluded [Gabeta, Norman, Liaskos, Papamichalis, Zografos, Garagounis, Rigopoulou and Dalekos (2007) J Clin Immunol 27: 378-87]. In another study, it was shown that the MIT3 assay, for instance, lacks all the necessary mitochondrial autoantigens for maximum diagnostic sensitivity of PBC [Dahnrich, Pares et al. (2009) Clin Chern 55: 978-85].

This highlights the need for the discovery and validation of additional autoantigen biomarkers to be used in solid-phase immunoassays for the optimal diagnosis of autoimmune diseases such as PBC. The most effective methods for the discovery of autoantigens are proteomics based. Proteomics can be defined as the global (e.g. parallel or simultaneous) analysis of the entire expressed protein compliment of the genome [Wasinger, Cordwell et al. (1995) Electrophoresis 16: 1090-4]. Proteomics methods allow for the discovery of novel autoantigens in an unbiased fashion. Common proteomics methods for discovery of novel autoantigens include SEREX (serological identification of antigens by recombinant expression cloning) [Krebs, Kurrer, Sahin, Tureci and Ludewig (2003) Autoimmun Rev 2: 339-45] and human proteome microarrays (“chips”, commonly the dimensions of standard microscope slides, containing thousands of purified recombinant human proteins printed to their surface in an ordered array of microscopic spots, e.g. spots of 100 micron in diameter) [Robinson, DiGennaro et al. (2002) Nat Med 8: 295-301; Robinson, Steinman and Utz (2002) Arthritis Rheum 46: 885-93].

### SUMMARY OF THE INVENTION

The present invention relates to methods of using the novel autoantigens (Tables I and V) human hexokinase 1 (HK1) and/or kelch-like 12 (KLHL12), or fragments thereof comprising an epitope, in the diagnostic, prognostic, staging and therapeutic regimens of the autoimmune liver disease Primary Biliary Cirrhosis (PBC). The present invention also relates to methods of using homologs, family members, transcript variants and isoforms (e.g. Table VI), preferably at least 70% identical, more preferably at least 90% identical and most preferably at least 95% identical, of human hexokinase 1 (HK1) and/or kelch-like 12 (KLHL12), or fragments thereof comprising an epitope, in the diagnostic, prognostic, staging and therapeutic regimens of the autoimmune liver disease Primary Biliary Cirrhosis (PBC).

The present invention further provides isolated antibodies that bind specifically to the above-described polypeptides, or fragments thereof comprising an epitope. Antibodies provided herein may be polyclonal or monoclonal, may be affinity purified, may be immobilized onto a solid support, and may be detectably labeled. The invention also provides methods for detecting the presence of an autoimmune disease in an animal, preferably a human, comprising the steps of isolating a body fluid sample, preferably blood, serum or plasma, from the animal, incubating the serum with an isolated HK1 and/or KLHL12 polypeptide described above, and detecting the binding of autoantibodies in the serum sample to the isolated polypeptide. The invention also provides alternative methods for detecting the presence of an autoimmune disease in an animal comprising the steps of isolating a body fluid sample from the animal, preferably blood, serum or plasma, and immobilizing components of the serum on a solid support, contacting the immobilized serum components with an isolated polypeptide described above under conditions favoring the formation of a complex between the serum components and isolated polypeptide, contacting the formed complex with an antibody that binds specifically to HK1 and/or KLHL12, and detecting the binding of the antibody to the complex. Autoimmune diseases that may be diagnosed by the methods of the present invention include primary biliary cirrhosis (PBC) and systemic lupus erythematosis (SLE). Cancers that may be diagnosed by the methods of the present invention include colorectal cancer (CRC). The present invention also provides methods of determining prognosis, disease stage and treatment regimens using the aforementioned methods of detecting autoantibodies against HK1 and/or KLHL12.

In a preferred embodiment, heterogeneous or homogenous immunoassays, singleplex or multiplex, are used to detect autoantibodies present in body fluids directed against said autoantigens. Other preferred embodiments of the present invention will be apparent to one of ordinary skill in light of the following drawings (Figures) and description of the invention, and of the claims.

### EXPERIMENTAL

### Example 1: Proteome Microarray Based Discovery of Novel Primary Biliary Cirrhosis (PBC) Autoantigens

### Serum Screening on Microarrays

Patient sera were screened against commercial human proteome microarrays comprised of ˜8,000 unique human recombinant (eukaryotically expressed) proteins printed in duplicate at high density to a “chip” the size of a standard microscope slide (HUMAN PROTOARRAY® v4.0, Invitrogen, Carlsbad, CA) [Sheridan (2005) Nat Biotechnol 23: 3-4]. Microarrays were performed according to the manufacturer's instructions. Microarrays were imaged on an ARRAYWORX®E BioChip fluorescence reader (Applied Precision, LLC, Issaquah, Washington) using the appropriate standard built-in filter sets. Image analysis and data acquisition was performed using the GENEPIX ® Pro v6.1 software package (Molecular Devices, Sunnyvale, CA) according to the instructions of the microarray manufacturer (HUMAN PROTOARRAY ® v4.0, Invitrogen, Carlsbad; CA).

92 different serum samples from normal individuals and patients with various diseases were individually screened against the proteome microarrays in order to detect the presence of autoantibodies against the arrayed proteins (potential autoantigens). For this, 2 different lots of microarrays were used in 2 sequential studies. The composition of the entire patient population was as follows: Microarray Lot #1 (80 unique samples)—18 Primary Biliary Cirrhosis (PBC) patients versus 62 non-PBC control samples [13 normal, colorectal cancer (CRC), 22 systemic lupus erythematosus (SLE), 2 Sjogrens syndrome (SjS)]. Microarray Lot# 2 (12 unique samples)—3 more PBC and 9 more non-PBC controls [4 normal and 5 autoimmune hepatitis (AIH)]. The normal sera were approximately age and gender matched to the PBC cohort. The AIH sera were used because it is an autoimmune liver disease different from PBC yet known to be associated with autoantibodies. The CRC sera were used because cancer patients are also known to have various autoantibodies against so-called tumor associated autoantigens (TAA), including a common repertoire of nuclear autoantibodies observed in both cancers and autoimmune disease [Bei, Masuelli, Palumbo, Modesti and Modesti (2008) Cancer Lett]. Archived sera were obtained from the repositories of the following sources: Our collaborator, Dr. Donald Bloch, M.D., Center for Immunology and Inflammatory Diseases, Massachusetts General Hospital, Assistant Professor of Medicine, Harvard Medical School provided 12 of the SLE sera as well as the SjS and PBC sera. Remaining SLE sera and all the AIH sera were from Bioreclamation Inc. (Hicksville, NY), normal sera were from ProMedDx, LLC (Norton, MA) and CRC sera were from Asterand Inc. (Detroit, MI).

### Biostatistical Analysis of Microarray Data

In order to identify the autoantigen biomarkers from the microarray data, the biostatistical methods used were the standard approaches provided by the microarray manufacturer in the form of the ProtoArray® Prospector v4.0 software package (Invitrogen, Carlsbad, CA) using the Immune Response Profiling (IRP)add-on [Hudson, Pozdnyakova, Haines, Mor and Snyder (2007) Proc Natl Acad Sci USA104: 17494-9]. Two of the biostatistical methods from this software package were used to create two corresponding PBC autoantigen lists as follows:

“Hit Calling” Autoantigen List: To convert the data to binary format, proteins (i.e. potential autoantigens) on each microarray (1 serum/microarray) were scored as a “hit” (i.e. positive) or not a hit (i.e. negative). Autoantigen hits were called on a per microarray basis using the Z-score with a cutoff threshold of 3 standard deviations above the microarray mean. The number of hits in the PBC and control groups for each autoantigen were used to determine the percent prevalence of each autoantigen. Autoantigens ultimately placed on this list had to have greater percent prevalence in the PBC cohort than the control cohort (i.e. all non-PBC samples).

M-Statistics Autoantigen List: This approach uses quantile normalized microarray data and performs a pairwise t-test for each protein between the two patient groups (i.e. PBC group and the control group corresponding to all non-PBC patients). This algorithm also estimates the autoantigen prevalence based on cutoffs set by the quantile normalized data. Autoantigens ultimately placed on this list had to have greater percent prevalence in the PBC cohort than the control cohort (i.e. all non-PBC samples) and had to have M-Statistics p-values of <0.1.

Microarray Lots # 1 and 2 were analyzed separately. To comprise a single final list of microarray-derived PBC autoantigens, those observed as overlapping on both aforementioned biostatistical lists for Microarray Lot #1 (only) were taken. Next, any markers on this compiled list that were positive in any of the AIH patients (Microarray Lot # 2), as determined by the “Hit Calling” method, were eliminated. Finally, the list was then prioritized based on the M-Statistics p-value as well as diagnostic sensitivity and specificity.

### Results:

Two of the PBC autoantigen markers, human Hexokinase 1 (HK1) and human Kelch-Like 12 (KLHL12), identified from the proteome microarrays and claimed in this patent, are listed in Table I, along with their M-Statistics p-values as well as their diagnostic sensitivities and specificities (calculated from Microarray Lot #1). Quantile normalized microarray data (normalized autoantibody signal intensity) for all 92 samples (i.e. all 92 microarrays) are shown in FIG. 16 and FIG. 17 for HK 1 and KLHL12 respectively. In summary (Table I), the presence of serum autoantibodies against either autoantigen is strongly correlated with the PBC cohort, showing highly significant p-values (1×10⁻¹⁰ and 8×10⁻⁵ for HK1 and KLHL12 respectively) as well as sensitivities of 85-89% and 33-40% for HK1 and KLHL12 respectively, and, specificities 84-90% and 97-98% for HK1 and KLHL12 respectively (see Table I for details). By definition (see “Biostatistical Analysis of Microarray Data” above in this Example), none of the 5 Autoimmune Hepatitis (AIH) sera were positive for HK1 or KLHL12 (see also FIG. 16 and FIG. 17; Microarray Lot #2). The HK1 and KLHL12 autoantigen biomarkers were also the subject of further validation as detailed in other experimental Examples.

It should also be noted that HK1 autoantibodies are also observed with low prevalence in systemic lupus erythematosis (SLE) and colorectal cancer (CRC) (FIG. 16). N-03 is the only “normal” serum sample to be positive for HK1 (FIG. 16; red bar). N-03 is also the only “normal” serum sample to be positive for KLHL12 (FIG. 17; red bar). Thus, in fact, it is believed that N-03 may in fact have yet undiagnosed or unreported/undocumented PBC (note that autoantibodies have been shown to pre-date clinical symptoms/manifestations of autoimmune disease, including in PBC).

### Example 2: Pre-Validation of Novel Primary Biliary Cirrhosis (PBC) Autoantigens HK1 and KLHL12 Using an ELISA

It should be noted that the ELISA assay described here in this Example and used in many subsequent Examples is termed T²-ELISA, and is based on the use of dual-epitope tagged cell-free expressed protein antigens. In this Example, those antigens are HK1 and KLHL12 and the T²-ELISA used as a tool for clinical pre-validation (and eventually validation in later Examples) of these microarray-derived novel autoantigens.

### Autoantigen Expression

The entire Open Reading Frames (ORFs) of human HK1 and KLHL12 were cloned, using standard and accepted molecular biology practices, into a plasmid vector compatible with cell-free protein expression, containing the T7 RNA polymerase promoter, a Kozak (ribosome binding) sequence, a start codon, an N-terminal VSV-G epitope tag (YTDIEMNRLGK(SEQ ID NO: 19)), and a C-terminal HSV epitope tag (QPELAPEDPED(SEQ ID NO: 20)) in addition to the ORF insert. As source DNA for cloning into the expression vector, full-length sequence-verified clones were purchased from OpenBiosystems (Huntsville, AL) [catalog OHS1770-9381021 (UniGene Hs.370365) for HK1 and MHS1011-61211 (UniGene Hs.706793) for KLHL12]. Expression vectors were verified for the correct ORF insert using standard EcoRI digestion methods and/or DNA sequencing.

Autoantigens were produced from the aforementioned plasmid clones by cell-free protein expression. Cell-free protein expression reactions were performed using a transcription/translation coupled rabbit reticulocyte lysate system (TNT® T7 Quick for PCR DNA; Promega, Madison, WI) according to the manufacturer's instructions. Autoantigen expression reactions contained the cognate plasmid DNA while blank expression reactions lacked only the plasmid DNA. Expression reactions were stopped by diluting 1/20 in TDB [1% BSA (w/v) and 0.1% (v/v) Triton X-100 in TBS-T (50 mM Tris, pH 7.5, 200 mM NaCl, 0.05% (v/v) Tween-20)].

### Dual-Tag Enzyme-Linked Immunosorbent Assay (T²-ELISA) of Autoantigens

NUNC-IMMUNO™ MICROWELL™ POLYSORP® 96 well white opaque, flat bottom, untreated polystyrene microtiter plates (Nunc Brand from Thermo-Fisher Scientific, Rochester, NY) were used for a sandwich type Enzyme-Linked Immunosorbent Assay (ELISA). Plates were coated with 0.5 μg/mL of a mouse monoclonal ANTI-HSV TAG ® capture antibody (EMD Biosciences, Inc., San Diego, CA) in sodium carbonate/bicarbonate pH 9.3 for 30 min with shaking (50 μL/well). Plates were then washed 6× in TB S-T (wells filled to maximum) on an ELx405 Select Robotic Plate Washer (BioTek, Winooski, VT). All plate washes were performed in this manner unless noted otherwise. Plates were then blocked for 30 min at 300 μL/well in 1% BSA (w/v) in TBS-T. The solution was removed from the plates and the aforementioned stopped (i.e. diluted) cell-free expression reactions (autoantigen and blank reactions) were then added at 100 μL/well and shaken for 30 min. Plates were washed and serum samples (diluted at 1/1,000 in 1% BSA (w/v) in TBS-T) were added at 100 μL/well and shaken for 30 min. Each serum sample was run against triplicate wells of autoantigen and triplicate wells of the cell-free expression blank. Additionally, one set of triplicate wells of autoantigen and one set of triplicate wells of the cell-free expression blank were designated for VSV-G epitope tag detection, and therefore received plain 1% BSA (w/v) in TBS-T instead of diluted serum. To avoid contamination of the robotic plate washer with human serum, plates were subsequently washed 4× by manual addition of TBS-T (wells filled to maximum) followed by vacuum aspiration and then washed 6× in the robotic plate washer as described earlier in this Example. Wells designated for detection of the VSV-G epitope tag then received an anti-VSV-G horseradish peroxidase (HRP) labeled monoclonal antibody (Clone P5D4, Roche Applied Science, Indianapolis, IN) diluted 1/20,000 in 1% BSA/TBS-T. Wells designated for detection of serum autoantibody received a mouse anti-[human IgG] HRP labeled monoclonal secondary antibody (minimum cross-reactivity with mouse immunoglobulin; Jackson ImmunoResearch Laboratories, Inc, West Grove, PA) diluted 1/20,000 in 1% BSA/TBS-T. Plates were shaken for 30 min. The solutions were then manually dumped from the plates by inversion followed by vigorous patting of the plates inverted on a dry paper towel to remove residual fluid. Plates were then washed in the robotic plate washer as described earlier in this Example. Chemiluminescence signal was generated by the addition of 50 μL/well of SUPERSIGNAL™ ELISA Pico Chemiluminesence Substrate (Pierce Brand from Thermo Fisher Scientific, Rockford, IL). Plates were developed by shaking for 15 min and then read on a LUMICOUNT™ luminescence plate reader (1 s exposure, PMT of 650V, gain 1) (Packard/PerkinElmer Life and Analytical Sciences, Inc., Boston, MA).

### Results:

For this pre-validation of the new PBC autoantigen markers listed in Table I, randomly selected sera that were detected as positive or negative for a given autoantigen in the microarray analyses (see Example 1) were also analyzed here by T² -ELISA.

Calculation of Autoantibody Units from the T²-ELISA, in short, was achieved by background subtracting the data and normalizing to the detection of the common VSV-G epitope tag for each antigen on each assay (i.e. each plate). More specifically, for each serum-autoantigen pair, for each of the triplicate wells from the T²-ELISA data, Autoantibody Units were calculated as follows: [autoantibody signal from one well (i.e. serum versus autoantigen)] minus [average background from triplicates (i.e. same serum versus average of all three blank expression wells)] to yield triplicate Background Subtracted Values (BSV) for each serum-autoantigen pair. Note that one assay is defined as one 96-well microtiter ELISA plate. To normalize for inter-assay variances (day-to-day and assay-to-assay) for each autoantigen, wells on each assay, for each autoantigen on that assay, were dedicated solely for detection of the common VSV-G epitope tag. The VSV-G Normalization Factor (VNF) was calculated as follows: [average VSV-G signal for triplicate wells (i.e. autoantigen wells probed with VSV-G antibody)] minus [average VSV-G background for triplicate wells (i.e. blank expression wells probed with VSV-G antibody]. On a per assay basis, the triplicate BSV for all serum-autoantigen pairs were then divided by the VNF for that assay and multiplied by 100, yielding triplicate Autoantibody Unit values for each serum-autoantigen pair (i.e. expressed as a percent of the VNF). Note that a floor of zero was set for the Autoantibody Units. The average and standard deviation (errors bars) were calculated and plotted in FIGS. 1 and 2 for the new PBC autoantigens HK1 and KLHL12 respectively.

Sera were scored “analytically”, as positive or negative in the T²-ELISA in order to check concordance with the microarrays. For this, both of the following criteria must have been met for each serum-autoantigen pair to have been scored as analytically positive in the T²-ELISA: i) a p-value≤0.05 in a 1-tailed homoscedastic unpaired t-test on the raw T²-ELISA values from the triplicate wells of the autoantibody signal (i.e. serum versus autoantigen) compared to background (i.e. same serum versus blank expression wells); ii) autoantibody signal-to-background ratio In FIGS. 1 and 2, T²-ELISA scores and microarray (“Array”) scores are denoted as positive (+) or negative (−). For HK1 (FIG. 1), of 12 randomly selected sera that were positive by the microarray analyses, 10 were positive by ELISA for 83% concordance. Additionally for HK1 (FIG. 1), 5 sera were randomly selected that were negative on the microarrays, all of which were also negative by T²-ELISA for a 100% concordance. For KLHL12, of the 7 negative and 4 positive sera randomly chosen from the microarray analyses (see Example 1), there was full 100% concordance with the T²-ELISA results as shown in FIG. 2.

### Example 3: Validation of Novel Primary Biliary Cirrhosis (PBC) Autoantigens HK1 and KLHL12 Using an ELISA on a New AMA-Positive PBC Patient Cohort Not-Previously Screened by Microarrays

### Autoantigen Expression

As in Example 2.

### Dual-Tag Enzyme-Linked Immunosorbent Assay (T² ELISA) of Autoantigens

As in Example 2.

### Results:

A critical validation of the newly discovered markers is to perform studies on a new patient cohort (22 PBC samples), never before screened on the proteome microarrays. In this Example, this has been done with both of the new PBC autoantigens, HK1 and KLHL12 (previously listed in Table I).

The new PBC sera were obtained from our collaborator, Dr. Donald Bloch, M.D., Center for Immunology and Inflammatory Diseases, Massachusetts General Hospital, Assistant Professor of Medicine, Harvard Medical School and the normal sera were from ProMedDx, LLC (Norton, MA).

Calculation of Autoantibody Units from the T²-ELISA, in short, was achieved by background subtracting the data and normalizing to the positive control on each assay (i.e. each plate), whereby the positive control is set to 1,000 Autoantibody Units. More specifically, for each serum-autoantigen pair, for each of the triplicate wells from the T²-ELISA data, Autoantibody Units were calculated as follows: [autoantibody signal from one well (i.e. serum versus autoantigen)] minus [average background from triplicates (i.e. same serum versus average of all three blank expression wells)]. This yields triplicate Background Subtracted Values (BSV) for each serum-autoantigen pair. Note that one assay is defined as one 96-well microtiter ELISA plate. To normalize for inter-assay variances (day-to-day and assay-to-assay) for each autoantigen, a common positive control PBC serum for HK1 and KLHL12 was run on every assay (selected from the microarray PBC cohort in Example 1). The positive control T²-ELISA data were processed in the aforementioned manner on a per assay basis and the triplicate BSV averaged to yield the Positive Control Normalization Factor (PCNF) for each assay. On a per assay basis, the triplicate BSV for all serum-autoantigen pairs were then divided by the PCNF for that assay and multiplied by 1,000, yielding triplicate Autoantibody Unit values for each serum-autoantigen pair. Importantly, the VSV-G common epitope tag detection (Example 2) was still used to verify successful and consistent autoantigen expression, but was not used here in the calculation of Autoantibody Units.

In order to set diagnostic scoring thresholds for a given autoantigen, the T²-ELISA assay was run on a group of 22 normal patient sera and the cutoffs then set at 2 standard deviations above the mean for this normal cohort, for ˜95% statistical confidence. The use of this method at 2-3 standard deviations is common practice (e.g. [Liu, Wang, Li, Xu, Dai, Wang and Zhang (2009) Scand J Immunol 69: 57-63]). However, a critical requirement of this standard deviation based cutoff calculation method is that the data follows a Gaussian distribution, yet a Shapiro-Wilk test for normality determined this was not the case. As a solution, we logy transformed the Autoantibody Units and set the floor to 0 (i.e. non-transformed values of ≤0 were left as 0 without transformation) yielding a Gaussian distribution (of the >0 values) and allowing cutoffs to be set based on the aforementioned standard deviation methodology. Autoantibody Unit values of ≤0 were excluded from the cutoff calculations because background subtraction is used in the calculation of Autoantibody Units, meaning patient samples yielding≤0 values would by definition have to be scored as autoantibody negative regardless (i.e. a cutoff is not needed nor relevant to ≤0 values).

As seen by the data in FIG. 3 for HK1, using a cutoff of 2.0, an 82% diagnostic sensitivity (100% specificity) on this new sample cohort is in good agreement with the microarray analyses performed on the original sample cohort (see Table I). As seen by the data in FIG. 4 for KLHL12, using a cutoff of 2.5, a 36% diagnostic sensitivity. (100% specificity) on this new sample cohort is in good agreement with the microarray analyses performed on the original sample cohort (see Table I).

### Example 4: Validation of Novel Primary Biliary Cirrhosis (PBC) Autoantigens HK1 and KLHL12 Using an ELISA on a New Anti-Mitochondrial Antibody (AMA)-Negative PBC Patient Cohort Not Previously Screened by Microarrays

Patients with suspected PBC but an antimitochondrial antibody (AMA)-negative status make up approximately 5-20% of all PBC patients [Oertelt, Rieger et al. Hepatology 2007; 45:659-665], and AMA-negative PBC patients are particularly difficult to confirm diagnostically based on serotesting. Employing the known and validated autoantigens Sp 100 and gp210 only results in the detection of a fraction of the AMA-negative PBC patients (e.g. 17-33% in one recent study [Liu, Shi, Zhang, Zhang and Gao (2008) Liver Int 28: 233-9]), showing a need for specific autoantigens which can detect AMA-negative PBC patients.

To test the ability of our novel autoantigens, HK1 and KLHL12, to detect AMA-negative PBC patients, we utilized 17 patient sera which were AMA-negative by indirect immunofluorescence (IIF) but with confirmed PBC by conventional methods [Heathcote (2000) Hepatology 31: 1005-13], and by liver biopsy. The new AMA-negative PBC sera were obtained from our collaborator, Dr. Donald Bloch, M.D., Center for Immunology and Inflammatory Diseases, Massachusetts General Hospital, Assistant Professor of Medicine, Harvard Medical School. We compared the ability of our novel autoantigens, HK1 and KLHL12, with the available commercial tests to detect these patients with confirmed PBC but a known AMA-negative status.

### Autoantigen Expression

As in Example 2.

### Dual-Tag Enzyme-Linked Immunosorbent Assay (T2-ELISA) of Autoantigens

As in Example 2.

### FDA-Approved Commercial PBC ELISAs

FDA-approved commercial ELISAs for PBC diagnostics were also run and were the QUANTA LITE™ M2 EP (MIT3), QUANTA LITE™ sp100, QUANTA LITE™ gp210 and QUANTA LITE™ PBC Screen IgG/IgA assays from INOVA Diagnostics (San Diego, CA); and were performed according to the manufacturer's instructions.

### Results:

For scoring purposes, Autoantibody Unit calculations and diagnostic thresholds established in Example 3 were once again employed here for each autoantigen (HK1 and KLHL12).

As illustrated by the data in FIG. 5 for HK1, 4 out of 17 AMA-negative PBC sera tested positive for this autoantigen (24% sensitivity). As seen by the data in FIG. 6 for KLHL12, 6 of the 17 AMA-negative PBC sera tested diagnostically positive (35% sensitivity).

We also tested the aforementioned 17 AMA negative PBC sera on all four of INOVA Diagnostics' commercially available FDA-approved PBC tests, namely, QUANTA LITE® ELISA assay for M2 EP (MIT3), QUANTA LITE® ELISA assay for sp100, QUANTA LITE® ELISA assay for gp210 and QUANTA LITE® ELISA assay for PBC Screen IgG/IgA ELISA. The results of these tests, as well as our T²-ELISA results with HK1 and KLHL12, are summarized in Table II. INOVA's tests were unable to detect 3 of the 17 patients (18%). Strikingly however, HK1 and KLHL12 were each able to detect one of the previously undetectable AMA-negative PBC sera (PB-AMN-044 and PB-AMN-263 respectively). The third patient (PB-AMN-084) remained undetected by the aforementioned autoantigens but was detected by Sp140 (see Example 6 for details). These results are summarized in FIG. 7 as a Venn Diagram, illustrating overlap (or lack thereof) between the various biomarkers. Note that the results of the QUANTA LITE® ELISA assay for PBC Screen IgG/IgA ELISA are not shown in the Venn Diagram (FIG. 7), however, as seen in Table II, this assay did not increase detection as compared to the other INOVA assays. Together, these findings indicate that our two novel autoantigens, HK1 and KLHL12, are diagnostically very significant. It suggests that adding our novel biomarkers to the existing panel of PBC biomarkers could result in. the improved detection, and therefore earlier treatment and improved outcome of PBC patients, in particular for AMA-negative PBC patients.

### Example 5: Assessing HK1 and KLHL12 in Patients with Atypical Indirect Immunofluorescent (IIF) Staining

We propose that the number of PBC patients may be higher than previously suspected, due to the extreme difficulty in drawing a conclusive diagnosis of PBC in the absence of definitive AMA staining or the proper anti-nuclear autoantibody (ANA) staining pattern as determined by indirect immunofluorescence (IIF). To test this theory, we examined sera from undiagnosed patients with diffuse cytoplasmic or nuclear membrane IIF staining patterns. These new patient sera were obtained from our collaborator, Dr. Donald Bloch, M.D., Center for Immunology and Inflammatory Diseases, Massachusetts General Hospital, Assistant Professor of Medicine, Harvard Medical School.

### Autoantigen Expression

As in Example 2.

### Dual-Tag Enzyme-Linked Immunosorbent Assay (T²-ELISA) of Autoantigens

As in Example 2.

### QUANTA LITE® ELISA Assay for M2 EP (MIT3)

Assay was performed according to manufacturer's instructions (INOVA Diagnostics, San Diego, CA).

### Results:

We ran HK1, KLHL12 and the M2 EP (MIT3) QUANTA LIT® Assay (INOVA Diagnostics, San Diego, CA) on 20 patients, the results of which are shown in FIG. 8. Serum samples prefixed with “Cyto” or “NM” are from patients with diffuse cytoplasmic or nuclear membrane IIF staining, respectively. Calculation of Autoantibody Units for the T²-ELISA as run on HK1 and KLHL12 was done as in Example 2. Scoring for the T²-ELISA assay was done according to the “analytical” method described in Example 2 (note that any serum sample with a graphed bar in FIG. 8 is positive). To avoid scale effects, graphed data for each antigen in FIG. 8 is normalized as a percent of the patient having the maximum autoantibody units for that antigen (that patient is marked with a blue arrow for each antigen). We set the Y-axis to INOVA' s MIT3 cut-off of 25 units (based on the low positive control; cutoff determined per manufacturer's instructions), which corresponded to 17%, so all bars shown represent positive results.

One patient is detected by all three markers. Novel autoantigen KLHL12 detects two nuclear membrane patients that no other markers detect. Finally, MIT3 detects one nuclear membrane and several cytoplasmic patients that no other marker detects. These results strongly suggest that detection of the HK1, KLHL12 and MIT3 antigens may be useful in revealing a large number of previously undiagnosed patients suffering from PBC, but with atypical IIF staining.

### Example 6: Improved Diagnostic Sensitivity by ELISA for Primary Biliary Cirrhosis (PBC) by Detection of Sp140

Antinuclear antibodies reacting with 5-20 nuclear dots are detected in 20-30% of patients with primary biliary cirrhosis (PBC). The “multiple nuclear dot” (MND) staining pattern produce by these antibodies is directed against promyelocytic leukemia protein nuclear body (PML NB) components, one of which was recently identified as Sp140. Sp140 has been reported to be present in 13% of PBC patients, with a larger proportion of AMA-negative compared with AMA positive PBC patients (53% versus 8%) [Granito, A. Yang, W. et. al, 2009, Am J Gastroenterol, In Press]. We therefore tested Sp140 in our T²-ELISA.

The PBC patient sera were obtained from our collaborator, Dr. Donald Bloch, M.D., Center for Immunology and Inflammatory Diseases, Massachusetts General Hospital, Assistant Professor of Medicine, Harvard Medical School. Sp140 status was initially determined by IIF on Sp140 expressing cells versus negative cells.

### Autoantigen Expression

As in Example 2.

### Dual-Tag Enzyme-Linked Immunosorbent Assay (T2-ELISA) of Autoantigens

As in Example 2.

### QUANTA LITE® ELISA Assay for Sp100 ELISA

Assay was performed according to manufacturer's instructions (INOVA Diagnostics, San Diego, CA).

### Results:

T²-ELISA Autoantibody Unit calculations and “analytical” scoring were performed as in Example 2. Scoring for the INOVA Diagnostics Sp100 ELISA were performed according to the manufacturer's instructions. Results are in Table III. Notably, although Sp100 was unable to be detected in PBC patients PB-AMP-020 or PB-AMN-084 (orange shading) by either our T²-ELISA or INOVA's assay, the T²-ELISA platform was able to detect these PBC patients using the Sp140 autoantigen. The detection of PB-AMN-084 is most notable, since this patient was not detected by any of the following: the Sp140 indirect immunofluorescence (IIF) methods (not shown)) any of INOVA's available PBC ELISA tests, or either of the novel autoantigens HK1 and KLHL12 as determined by T²-ELISA (see earlier in Example 4 and Table II for these ELISA results).

Together then, HK1, KLHL12 and Sp140 may serve as a powerful diagnostic panel of autoantigens which enable the rapid and accurate diagnosis of previously missed PBC patients.

This Example also demonstrates another important result, that is, with respect to Sp100, our T²-ELISA platform is essentially 100% concordant with INOVA's FDA approved Sp100 ELISA. The only discordant results were 2 cases where the T²-ELISA gave a negative result and the INOVA assay an equivocal result, that is, too close to INOVA's designated cutoff to be conclusive (per the manufacturer's scoring methods).

### Example 7: Colorimetric Versus Chemiluminescent ELISA Detection of Autoantibodies Against the Novel Primary Biliary Cirrhosis (PBC) Autoantigens HK1 and KLHL12 Using PBC Patient Serum

ELISA experiments exploring the binding between autoantigens and autoantibodies usually employ one of two detection strategies. Chemiluminescence is generally accepted to be more sensitive and has a broader dynamic range, while colorimetric is generally accepted to be more stable and consistent. The purpose of these experiments was to perform the exact same experiment twice and then to develop it in parallel, once by colorimetric detection, and once by chemiluminescent detection.

### Autoantigen Expression and T2-ELISA

Performed as in Example 2 except that for the colorimetric ELISA detection, the following reagents from the INOVA Diagnostics QUANTA LITE® ELISA platform (San Diego, CA) were utilized: HRP Sample Diluent, HRP Wash Concentrate, HRP IgG Conjugate, TMB Chromogen, HRP Stop Solution. Instructions were followed per the manufacturer. The diagnostic scoring for the chemiluminescent ELISA were those as already determined in Example 4 for the same sera.

### Results:

ELISA results of HK1 on sera from PBC patients are shown in FIG. 9A and KLHL12 in FIG. 9B, demonstrating both colorimetric and chemiluminescent detection. Colorimetric assay results are plotted as signal minus background, with the background being the same serum run against an expression blank (no autoantigen expressed). The chemiluminescence ELISA score is listed under the X-Axis as “+” (positive) or “−” (negative). Note that the scores for the chemiluminescent ELISA were those as already determined in Example 4 for the same sera (with sera PB-AMN-044 and PB-AMN-263, green outline in FIGS. 9A and B, being the ones that scored previously negative for all available PBC ELISA assays from INOVA Diagnostics but positive for HK1 and KLHL12 respectively). These results clearly demonstrate concordance between the chemiluminescent and colorimetric ELISA readout methods.

### Example 8: Feasibility of Point-of-Care Diagnostics Colorimetric Dot Blot Detection of Autoantibodies Against the Novel Primary Biliary Cirrhosis (PBC) Autoantigen HK1 Using PBC Patient Serum

The purpose of this example is to show proof-of-principle for use of autoantigens in a point-of-care (POC) autoantibody based diagnostic assay for autoimmune disease (i.e. an assay that is rapidly and readily performed in the doctor's office, e.g. by an internist, general practitioner or rheumatologist).

One common format of a solid-phase immunoassay for point-of-care (POC) diagnostics is the lateral flow based immuno-chromatographic method, performed on a porous solid membrane matrix, such as nitrocellulose. For example, a blood sample as well as a colorimetrically labeled detector reagent (commonly a colloidal gold label) are allowed to flow by capillary action across the length of a nitrocellulose strip, subsequently contacting the test area where, for example, an antigen, capture antibody or other capture agent had been previously immobilized (i.e. striped). A positive result is visualized as a colored stripe in the test area.

The most ubiquitously recognized form of such an assay is the “home” pregnancy test, however, various formats for rapidly detecting antibodies in human blood, e.g. for detection of pathogen infection, are possible [Biagini, Sammons, Smith, MacKenzie, Striley, Snawder, Robertson and Quinn (2006) Clin Vaccine Immunol 13: 541-6; Laderman, Whitworth, Dumaual, Jones, Hudak; Hogrefe, Carney and Groen (2008) Clin Vaccine Immunol 15: 159-63].

To mimic this type of device and show feasibility with the new PBC autoantigen HK1 reported in this patent, a dot blot assay was performed. In this assay, autoantigen is immobilized on a nitrocellulose membrane which is then probed with patient serum. Detection of bound autoantibody is achieved with a colloidal-gold labeled anti-human IgG detector antibody. Details of the procedure and results are as follows:

### Colorimetric Dot Blot of Autoantigen

Recombinant purified human Hexokinase 1 protein (HK-1, Alpha Diagnostic, International, San Antonio, TX) was diluted to 200 ng/μL in TBS (50 mM Tris, pH 7.5, 200 mM NaCl). Human IgG was diluted to 250 ng/μL in PBS (50 mM sodium phosphate, pH 7.5, 100 mM NaCl).

Nitrocellulose (HiFlow Plus, Millipore Corporation, Bedford, MA) was cut to form 0.5 cm×3 cm strips. 1 μL each of TBS, HK1 and human IgG were individually spotted onto the nitrocellulose and allowed to dry thoroughly by incubation for 1 h at 37° C. Strips were then treated in Block buffer [1% BSA (w/v) in TBS-T (TBS with 0.05% v/v Tween-20)] for 30 min at room temperature (RT). Block was vacuum aspirated. Patient serum was diluted 1:100 in Block and then incubated with nitrocellulose strips for 30 min at RT. Serum was aspirated and the strips were washed with 1.5 mL TBS-T: 4×5 min each. Strips were probed with colloidal gold conjugated secondary antibody [Anti-Human IgG (H+L) antibody, Gold labeled (40 nm), KPL, Gaithersburg, MD] diluted 1:10 in Block shaking at RT for 3 hours.

### Results:

Lateral flow immunoassays offer a simple, accurate, fast result-reporting and ease-of-use format and thus are a popular point-of-care (POC) diagnostic platform. Lateral flow-based devices use immunochromatographic principles to assay bio-fluids such as blood for various analytes in a matter of minutes, under “field” conditions with no special instrumentation or expertise. To test the feasibility of a colorimetric lateral flow POC assay of PBC autoantigens, we performed a model dot blot experiment.

Recombinant purified human HK1 was spotted onto nitrocellulose, as well as. carrier buffer (negative control) and human IgG (positive control). Diluted sera (1:100) from a PBC patient and normal patient was allowed to bind and washed before adding colloidal gold labeled anti-human IgG. Results are shown in FIG. 10. After 1 h 20 min, all IgG spots (positive controls) had turned pink. The HK1 spot turned pink with 1:100 dilution of PBC patient serum but was negative (no color) with normal serum. Negative control spots (carrier buffer only) remained colorless.

### Example 9: A Dual-Epitope Tag Based Solid-Phase Heterogeneous Assay (T²-ELISA) as a Tool for Detecting Protein Interactions

We have developed a novel, high throughput and internally normalized solid- phase heterogeneous assay which is based on dual-epitope tagged cell-free (in vitro) expressed target proteins captured on a surface. The assay can detect the binding of “probes” (e.g. drugs, oligonucleotides or antibodies) to the surface-immobilized cell-free expressed target proteins while being able to normalize for the amount of target protein on the same surface. Although the Example shown here relates to detection of autoantibody binding from human serum to cell-free expressed autoantigens as the target proteins, the methodology is broadly applicable. Furthermore, although the assay format used in this Example is a micro-well (microtiter) plate based ELISA format, various assay formats are possible.

One embodiment of our novel assay, which we shall call the T²-ELISA method, comprises the capture of an autoantigen (target protein) onto the microtiter plate well with one epitope tag (capture tag) followed by reading the autoantibody (probe) signal in the same well, while using the other tag (detection tag) to normalize for the amount of protein expressed in separate wells. In order to compare our T²-ELISA assay with an FDA-approved, commercially available, semi-quantitative ELISA assay for the detection of anti-sp100 IgG antibodies in human serum (QUANTA LITE ® ELISA assay for sp100; INOVA Diagnostics, San Diego, CA) we set up the following experiment: Briefly, autoantigens are cell-free expressed, purified in-line with the microtiter plate based assay (i.e. captured on well surface) and screened against patient sera for autoantibody binding using a traditional sandwich ELISA format. Enzyme-tagged detector antibodies (each having a different chemiluminescent substrate) are added in series, after which two different chemiluminescent substrates are added to the appropriate wells one at a time in order to read both autoantibody binding as well as the detection tag (normalization signal).

### Autoantigen Expression

The entire Open Reading Frame (ORF) of the putative autoantigen (in this case human Sp100) was cloned, using standard and accepted molecular biology practices, into a plasmid vector compatible with cell-free protein expression, containing the T7 RNA polymerase promoter, a Kozak (ribosome binding) sequence, a start codon, an N-terminal VSV-G epitope tag (YTDIEMNRLGK(SEQ ID NO: 19)), and a C-terminal HSV epitope tag (QPELAPEDPED(SEQ ID NO: 20)) in addition to the ORF insert. As source DNA for cloning into the expression vector, full-length sequence-verified clones were purchased from OpenBiosystems (Huntsville, AL). Expression vectors were verified for the correct ORF insert using standard EcoRI digestion methods.

Autoantigens were produced from the aforementioned plasmid clones by cell-free protein expression. Cell-free protein expression reactions were performed using a transcription/translation coupled rabbit reticulocyte lysate system (TNT® T7 Quick for PCR DNA; Promega, Madison, WI) according to the manufacturer's instructions: Autoantigen expression reactions contained the cognate plasmid DNA while blank expression reactions lacked only the plasmid DNA. Expression reactions were stopped by diluting 1/20 in TDB [1% BSA (w/v) and 0.1% (v/v) Triton X-100 in TBS-T (50 mM Tris, pH 7.5, 200 mM NaCl, 0.05% (v/v) Tween-20)].

### Enzyme-Linked Immunosorbent Assay (ELISA) of Autoantigens

NUNC-IMMUNO™ MICROWELL™ POLYSORP® 96 well white opaque, flat bottom, untreated polystyrene microtiter plates (Nunc Brand from Thermo-Fisher Scientific; Rochester, NY) were used for a sandwich type Enzyme-Linked Immunosorbent Assay (ELISA). Plates were coated with 0.5 μg/mL of a mouse monoclonal ANTI-HSV TAG ® capture antibody (EMD Biosciences, Inc., San Diego, CA) in sodium carbonate/bicarbonate pH 9.3 for 30 min with shaking (50 μL/well). All plate washing consisted of manual addition of TBS-T (wells filled to maximum, i.e. 300 μL) followed by vacuum aspiration, repeated 4x. All plate washes were performed in this manner unless noted otherwise. Plates were then blocked for 30 min at 300 μL/well in 1% BSA (w/v) in TBS-T. The solution was removed from the plates and the aforementioned stopped (i.e. diluted) cell-free expression reactions (autoantigen and blank reactions) were then added at 100 μL/well and shaken for 30 min. Plates were washed and serum samples (diluted at 1/1,000 in 1% BSA (w/v) in TBS-T) were added at 100 μL/well and shaken for 30 min. Plates were washed and serum samples (diluted at 1/1,000 in 1% BSA (w/v) in TBS-T) were added at 100 μL/well and shaken for 30 min. Each serum sample was run against duplicate wells of autoantigen and duplicate wells of the cell-free expression blank with an additional set of duplicate wells of the cell-free expression blank designated for VSV-G epitope tag detection [thus received plain 1% BSA (w/v) in TBS-T instead of diluted serum]. Wells designated for detection of the VSV-G epitope tag then received an anti-VSV-G horseradish peroxidase (HRP) labeled monoclonal antibody, while wells designated for detection of serum autoantibody received a mouse anti-[human IgG] HRP labeled monoclonal secondary antibody. Plates were subsequently washed 4× by manual addition of TBS-T (wells filled to maximum) followed by vacuum aspiration as described earlier in this Example. Wells designated for detection of the VSV-G epitope tag then received an anti-VSV-G horseradish peroxidase (HRP) labeled monoclonal antibody (Clone P5D4, Roche Applied Science, Indianapolis, IN) diluted 1/20,000 in 1% BSA/TBS-T. Wells designated for detection of serum autoantibody received a mouse anti-[human IgG] HRP labeled monoclonal secondary antibody (minimum cross-reactivity with mouse immunoglobulin; Jackson ImmunoResearch Laboratories, Inc, West Grove, PA) diluted 1/20,000 in 1% BSA/TBS-T. Plates were shaken for 30 min. The solutions were then manually dumped from the plates by inversion followed by vigorous patting of the plates inverted on a dry paper towel to remove residual fluid. Plates were then washed as described earlier in this Example. Chemiluminescence signal was generated by the addition of 50 μL/well of SUPERSIGNAL™ ELISA Pico Chemiluminesence Substrate (Pierce Brand from Thermo Fisher Scientific, Rockford, IL). Plates were developed by shaking for 15 min and then read on a LUMICOUNT^{TM} luminescence plate reader (1 s exposure, PMT of 650V, gain 1) (Packard/PerkinElmer Life and Analytical Sciences, Inc., Boston, MA).

QUANTA LITE® ELISA Assay for sp100

Assay was performed according to manufacturer's instructions (INOVA Diagnostics, San Diego, CA).

### Results:

We compared our T²-ELISA to a commercial ELISA to test concordance (FIG. 11). This was done by testing 35 primary biliary cirrhosis (PBC) sera for autoantibodies against the known autoantigen Sp100. The commercial ELISA (INOVA Diagnostics, San Diego, CA) is an FDA-approved colorimetric ELISA comprised of autoantigen immobilized on the plate surface and was performed according to the manufacturer's instructions. Data are shown in FIG. 11 using a subset of the PBC cohort. The INOVA standard positive control serum used to calculate “Units” was run on both assays to convert the signals of each assay to the same scale (Units/μL of Neat Serum). Both assays were scored using the INOVA methodology, i.e. positive when units>25; which is what the “Low Positive” standard positive control serum is set to. As FIG. 11 indicates, in terms of scoring sera positive or negative, there is perfect concordance. However, the INOVA assay saturates very quickly, while the T²-ELISA displays at least a 5-fold wider dynamic range.

### Example 10: Comparison of T²-ELISA with a Conventional Commercial ELISA for p53 Tumor Associated Autoantibody Detection From Cancer Sera in Order to Assess Concordance

### Autoantigen Expression for T²-ELISA

The entire Open Reading Frame (ORF) of human p53 was cloned, using standard and accepted molecular biology practices, into a plasmid vector compatible with. cell-free protein expression, containing the T7 RNA polymerase promoter, a Kozak (ribosome binding) sequence, and C-terminal HSV (QPELAPEDPED(SEQ ID NO: 20)) and 6× His epitope tags, in addition to the ORF insert. Expression vectors were verified for the correct ORF insert using DNA sequencing.

The p53 autoantigen was produced from the aforementioned plasmid clone by cell-free protein expression. Cell-free protein expression reactions were performed using a transcription/translation coupled rabbit reticulocyte lysate system (TNT® T7 Quick for PCR DNA; Promega, Madison, WI) according to the manufacturer's instructions. Autoantigen expression reactions contained the cognate plasmid DNA while blank expression reactions lacked only the plasmid DNA. Expression reactions were stopped by diluting 1/20 in TDB [1% BSA (w/v) and 0.1% (v/v) Triton X-100 in TBS-T (50 mM Tris, pH 7.5, 200 mM NaCl, 0.05% (v/v) Tween-20)].

### Enzyme-Linked Immunosorbent Assay (T²-ELISA) of Autoantigens

Sera (ProMedDx, Norton, MA) from 34 patients diagnosed with colorectal cancer (CRC) of varying stages (ranging from AJCC/UICC Stage Ito Stage IV) and from 7 disease-free individuals were screened in duplicate for autoantibodies against the p53 tumor autoantigen using a commercial ELISA (EMD Biosciences, Inc., San Diego, CA) comprised of recombinant human cellular expressed p53 and the T²-ELISA. For the commercial ELISA, sera, pre-cleared with a 5 minute spin at 16,000× g in a microcentrifuge at 4° C., were diluted 1:100 and run in duplicate following instructions provided by the manufacturer and described in the literature [Oshikawa and Sugiyama (2000) Respir Med 94: 1085-91]. A validated negative control sera (provided by the manufacturer) was also run in duplicate and used to determine assay background. Absorbance readings at 450 nm for each well were collected on a SpectraMax Plus384 microplate spectrophotometer (Molecular Devices, Sunnyvale, CA).

For screening sera with the T²-ELISA, the following protocol was used. NUNC-IMMUNO™ MICROWELL™ POLYSORP® 96 well white opaque, flat bottom, untreated polystyrene microtiter plates (Nunc Brand from Thermo-Fisher Scientific, Rochester, NY) were used for a sandwich type Enzyme-Linked Immunosorbent Assay (ELISA). Plates were coated with 0.5 μg/mL of a mouse monoclonal ANTI-HSV TAG ® capture antibody (EMD Biosciences, Inc., San Diego, CA) in sodium carbonate/bicarbonate pH 9.3 for 30 min with shaking (50 μL/well). Plates were then manually washed 4× in 300 μL TBS-T using a multichannel pipette to add the wash buffer and inversion of the plates followed by vigorous patting of the inverted plates on a dry paper towel to remove the wash buffer and residual fluid. Blocking was performed for 30 min with 300 μL/well in 1% BSA (w/v) in TBS-T. The solution was removed from the plates as just described and the aforementioned stopped (i.e. diluted) cell-free expression reactions (autoantigen and blank reactions) were then added at 100 μL/well and shaken for 30 min. Plates were washed as above and serum samples (pre-cleared with a 5 minute spin at 16,000×g in a microcentrifuge at 4° C.) were diluted at 1/2,000 in 1% BSA (w/v) in TBS-T. A volume of 100 μL serum/well was added and plates were shaken for 30 minutes at room temperature. Each serum sample was run against duplicate wells on each of two separate plates, one containing cell-free expressed autoantigen and the other containing cell-free expression blank (expression reaction minus DNA template). Following serum incubation, serum was removed by vacuum aspiration and plates were washed 4× with TBS-T. For serum autoantibody detection, 100 μ1 of a mouse anti-[human IgG] HRP labeled monoclonal secondary antibody (minimum cross-reactivity with mouse immunoglobulin; Jackson ImmunoResearch Laboratories, Inc, West Grove, PA) diluted 1/20,000 in 1% BSA/TBS-T was added to each well. Plates were shaken for 30 min at room temperature followed by washing 4× in 300μl TBS-T as described above. Chemiluminescence signal was generated by the addition of 50 μL/well of SUPERSIGNAL™ ELISA FEMTO Chemiluminesence Substrate (Pierce Brand from Thermo Fisher Scientific, Rockford, IL). Plates were developed by shaking for 15 seconds at room temperature and then read on a LUMICOUNT™ luminescence plate reader (1 s exposure, PMT of 693V, gain 1) (Packard/PerkinElmer Life and Analytical Sciences, Inc., Boston, “MA).

### Results:

To test concordance of our T²-ELISA with the commercial ELISA in detecting autoantibodies against p53, a known tumor autoantigen, 34 sera from CRC patients (FIGS. 12, 1-34) and 7 sera from disease-free, “normal” individuals [FIG. 12, N1-N7 (outlined by green box)] were tested in duplicate on each of the two assays. After running each ELISA, signal minus background values were first calculated for each sera. For the commercial ELISA, background was calculated as the average of the raw values from each of the two wells probed with a validated negative sera provided by the manufacturer. This background value was then subtracted from the raw values of each of the test wells probed with either CRC or “normal” sera, yielding duplicate signal-minus-background values for each sera. Note that a floor of zero was set for these signal-minus-background values (i.e. any negative values were set to zero). The duplicate signal-minus background values for each sera were then averaged yielding a single, average, signal-minus-background value. For the T²-ELISA, background was determined as the average of the duplicate wells for each serum run against the cell-free expression blank (minus DNA template reaction). This background value was then independently subtracted from each of the duplicate raw values for the same serum run against cell-free expressed autoantigen (p53) yielding two signal-minus-background values for each sera. As with the analysis of the commercial ELISA data, a floor of zero was once again set for the signal-minus-background values. The duplicate signal-minus-background values for each sera were then averaged yielding a single, average, signal-minus-background value for each sera. Next, for both the commercial ELISA and T²-ELISA, sera were simply scored as analytically positive or negative (FIG. 12 shows only those sera scored as analytically positive) in order to check concordance between the two assays. For this, both of the following criteria must have been met for each serum-autoantigen pair in order for that pair to have been scored as analytically positive in the ELISA: i) a p-value≤0.05 in a 1-tailed homoscedastic unpaired t-test on the raw ELISA values from the duplicate wells of the autoantibody signal (serum versus autoantigen) compared to values from the duplicate wells of the background signal (same serum versus blank expression wells); ii) autoantibody signal-to-background ratio≥2. Serum-autoantigen pairs not passing these criteria are set to 0. Finally, for each assay independently, the average signal-minus-background values of those sera scored as analytically positive were normalized to the serum with the highest value in that same assay (CRC 12 for the commercial ELISA and CRC19 for the T²-ELISA), which was set to 100%. These normalized values were then plotted with error bars representing standard deviations (FIG. 12). As can be noted in FIG. 12, all sera that scored positive for p53 autoantibodies in the commercial ELISA also scored positive (with an approximately equal relative strength of signal, also) in the T²-ELISA. Additionally, one additional CRC serum (serum 18), but no additional normal serum, was scored slightly positive by the T²-ELISA and negative by the commercial ELISA. Together, the data suggest that the T²-ELISA is at least as sensitive as the commercial ELISA, and perhaps may even be slightly more sensitive as indicated by the ability to identify one additional CRC sample. Neither assay detected an autoantibody signal in any of the normal sera, suggesting a very good concordance with respect to specificity also.

### Example 11: A Dual-Epitope Tag and Dual-Reporter Based Solid-Phase Heterogeneous Assay as a Tool for Detecting Interactions with Proteins

The dual-tagged T²-ELISA described in Example 2 utilizes a single-reporter system for autoantibody detection and target protein normalization. Whereas Example 2. demonstrates using separate wells for probe readout (autoantibody in that case) and epitope tag readout, this Example illustrates the ability of the assay to detect the binding of “probes” (e.g. drugs, oligonucleotides or antibodies) to the surface-immobilized cell-free expressed target proteins while being able to normalize for the amount of target protein on the same surface (i.e. same well), using a dual-reporter system. Although the Example shown here relates to detection of autoantibody binding from human serum to cell-free expressed autoantigens as the target proteins, the methodology is broadly applicable. Furthermore, although the assay format used in this Example is a micro-well (microtiter) plate-based ELISA format, various assay formats are possible.

In order to show that it is possible to capture an autoantigen (target protein) onto the microtiter plate well with one epitope tag (capture tag) and normalize with the other (detection tag), while still reading the autoantibody (probe) signal in the same well, we performed the T²-ELISA assay as described in Example 2, with the following exceptions: following cell-free expression and antigen capture, and the sequential addition of enzyme-tagged antibodies, two different chemiluminescent substrates were also added sequentially, thereby enabling both autoantibody binding signals and detection tag (normalization) signals to be read sequentially within the same well.

In addition to showing that dual detection within the same well is possible, we directly compare dual-well detection to single-well detection on a variety of autoantigens with various patient sera, in order to demonstrate the potential advantages of per-well-normalization, namely, by normalizing for possible protein expression or capture variations.

### Autoantigen Expression

Performed as in Example 2, with the exception of Rap55, which was expressed from column-purified PCR product. Rap55 was PCR-amplified from cDNA using standard and accepted molecular biology practices. Primers were designed to yield a PCR product compatible with cell-free protein expression, containing the T7 RNA polymerase promoter, a Kozak (ribosome binding) sequence, a start codon, an N-terminal VSV-G epitope tag (YTDIEMNRLGK(SEQ ID NO: 19)), and a C-terminal HSV epitope tag (QPELAPEDPED(SEQ ID NO: 20)) in addition to the Rap55 insert.

### Enzyme-Linked Immunosorbent Assay (T²-ELISA) of Autoantigens

Performed as in Example 2, with the following exceptions. For the dual-reporter assay (different from the single-reporter assay as described in Example 2) there were no additional wells set aside for VSV-G epitope tag detection, since the tag and the probe (autoantibody) were detected sequentially in the same well. The enzyme-tagged antibodies were added sequentially to all the wells, followed each time by washing, as described here: First a mouse anti-[human IgG] alkaline phosphatase (AP) labeled monoclonal secondary antibody (minimum cross-reactivity with mouse immunoglobulin; Jackson ImmunoResearch Laboratories, Inc, West Grove, PA) diluted 1/20,000 in 1%. BSA/TBS-T was added. Plates were then shaken for 30 min. The solutions were then manually dumped from the plates by inversion followed by vigorous patting of the plates inverted on a dry paper towel to remove residual fluid. Plates were then washed manually as described earlier in Example 8. This process was repeated for an anti-VSV-G horseradish peroxidase (HRP) labeled monoclonal antibody (Clone P5D4, Roche Applied Science, Indianapolis, IN) diluted 1/20,000 in 1% BSA/TBS-T. An AP chemiluminescence signal was generated by the addition of 50 μL/well of BM Chemiluminescence ELISA Substrate (Alkaline Phosphatase Detection; Roche Diagnostics, GmbH, Mannheim, Germany) following the manufacturer's instructions. After allowing the signal to develop, plates were read as described in Example 8, followed by a second reading where PMT was set relative to the highest signal on the plate. After reading the plate, the plate was washed manually followed by the addition of 50 μL/well of SuperSignal ELISA Pico Chemiluminescence Substrate (Pierce Brand from Thermo Fisher Scientific, Rockford, IL). Plates were developed by shaking for 15 min and then read as described in Example 1, followed by a second reading where PMT was set relative to the highest signal on the plate.

Different from the data in Table IV, the dual-reporter and single-reporter ELISAs performed for FIG. 13 were washed with the aid of a robotic plate washer. Specifically, Plates were washed 6× in TB S-T (wells filled to maximum) on an ELx405 Select Robotic Plate Washer (BioTek, Winooski, VT). Following the addition of serum, in order to avoid contamination of the robotic plate washer with human serum, plates were subsequently washed 4× by manual addition of TBS-T (wells filled to maximum) followed by vacuum aspiration and then washed 6× in the robotic plate washer as described earlier in this Example.

### Results:

First, in order to establish that the dual-detection process of the T²-ELISA is as efficient as single detection, we directly compared this using Rap55, a known PBC autoantigen, and a PBC patient serum sample. As seen in Table IV-A, the autoantibody (AP) signal [calculated as AP signal-noise (i e same serum versus blank expression wells)] from the dual-reporter assay was calculated as a percent of the corresponding autoantibody signal from the single-reporter (AP) assay. Both methods yielded almost identical results (dual reporter AP signal was 97% of corresponding single reporter, dual reporter HRP signal was 96% of corresponding single reporter), clearly demonstrating that detection of the VSV-G epitope tag (HRP) does not inhibit the subsequent detection of the autoantibody signal (AP) in the same well. Likewise, autoantibody (AP) detection does not significantly interfere with VSV-G epitope tag (HRP) detection in the same well. We also calculated signal-to-noise ratios for the autoantibody (AP) signal: [calculated as AP signal/noise (i.e. same serum versus blank expression wells)] from the dual-reporter assay as compared to the single-reporter assay (Table IV-B) and demonstrated that dual detection within the same well does not decrease the signal-to-noise ratios in the slightest.

Second, dual-reporter and single-reporter T²-ELISA assays were compared for several serum-antigen pairs. FIG. 13 shows example data from T² -ELISA for systemic lupus erythematosus (SLE), PBC and normal patient sera versus a variety of known autoantigens (CENPB, Ro-60, Smith B, and Sp140). As a reference, samples were already known to be positive for the various autoantigens as reported by clinical annotation of samples. Autoantibody Unit ELISA values were determined for each serum-autoantigen pair, for which the average and standard deviation (errors bars) was calculated and plotted in FIG. 13 individually for the aforementioned autoantigens. Note that a floor of zero was set for the Autoantibody Units. Normal sera tested with CENPB are indeed negative as expected. Signal-to-noise ratios of positive results ranged from 3:1 (Smith B vs. SLE-H) to 300:1 (SP140 vs. PBC-I-21). This experiment also compares the dual-reporter assay to a single-reporter assay whereby separate wells were used solely for the detection of the VSV-G normalization epitope tag. The potential advantage of dual-reporter detection is that each autoantibody signal is normalized per well for possible protein expression (e.g. day-to-day) or capture variations (intra- or inter-assay). The data shows no significant detriment to using the dual-reporter assay. Furthermore, as expected, standard deviations of the dual-reporter assay, which is a per-well normalization, are significantly less than the single-reporter assay, which normalizes only on a per assay (per plate) basis.

### Example 12: Detection of Autoantibodies Against the Novel Primary Biliary Cirrhosis (PBC) Autoantigens HK1 and KLHL12 Recombinantly Expressed in a Wheat Germ Based System and Assayed Using a Direct Autoantigen Coating to the Surface of the ELISA Plate

### Autoantigens and ELISA Assay

In this Example, a key feature is that the ELISA assay was performed on polystyrene microtiter plates directly coated with pre-purified recombinantly expressed autoantigens (instead of antibody mediated in situ capture/purification to ELISA plate surface as in T²-ELISA). Another notable feature is that HK1 and KLHL12 were expressed in a different system as compared to previous Examples. Human HK1 and KLHL12 full-length recombinant proteins expressed in a cell-free wheat germ based system and purified by their N-terminal GST fusion tag were purchased from Abnova (Taiwan). The plates were coated overnight with 100 μL per well of 0.5 μg/mL recombinant protein diluted in PBS. As detailed in Example 2, plates were then washed 6× in TBS-T (wells filled to maximum) and then were blocked for 30 min at 300 μL/well in 1% BSA (w/v) in TBS-T. The block solution was removed from the plates and serum samples (diluted at 1/100) (diluent from INOVA Diagnostics' QUANTA LITE ® ELISA system; San Diego, CA) were added at 50 μL/well and shaken for 30 min at room temperature. Plate washing and addition of the secondary antibody is described in Example 2. The ELISA was developed using the colorimetric substrate and stop solution from INOVA Diagnostics' QUANTA LITE® ELISA system (San Diego, CA) according to the manufacturer's instructions.

### Results:

FIG. 14 shows that the colorimetric assay works well for HK1 versus several PBC and normal sera and results are 100% concordant with the expected results (based on the microarray and T²-ELISA results; see Examples 1 and 2). Note these expected scores are indicated by “+” and “−” in the graph. Note that the red line is the cutoff for this assay (set at 2 standard deviations above the mean for the 4 expected negative samples). Also note that this is direct plate coating with a recombinant antigen and there is no background subtraction here (it is not needed with no capture antibody present). Finally, note that N-03 is in fact supposed to be positive (and PBC-04 and PBC-05 negative) based on previous results from Examples 1 and 2.

Similarly, FIG. 15 for KLHL12 shows colorimetric assay results that are 100% concordant with the expected results (based on the microarray and T²-ELISA results; see Examples 1 and 2). Note these expected scores are indicated by “+” and “−” in the graph. The cutoff is indicated as the red line and was set 2 standard deviations above the mean for the 4 expected negative samples. N-03 is expected to be positive and PBC-02 and PBC-07 negative based on previous results from Examples 1 and 2.

### Example 13: Detection of Autoantibodies in Primary Biliary Cirrhosis (PBC) Using Homologs of HK1 and KLHL12

Information in the following paragraphs was obtained from the publically available UniProt database [The-UniProt-Consortium (2009) Nucleic Acids Res 37: D169-74] as well as the various publically available NCBI databases [National (United States) Center for Biotechnology Information].

Hexokinase 1 (HK1) is a protein which localizes to the outer membrane of mitochondria. Alternative splicing the gene encoding HK1 results in five transcript variants which encode different isoforms. Each isoform has a distinct N terminus but the remainder of the protein is identical among all isoforms [NCBI RefSeq]. Therefore, it is reasonable to assume that any of the aforementioned isoforms would be sufficient for detection of autoantibodies to hexokinase 1 in Primary Biliary Cirrhosis (PBC).

Furthermore, Hexokinase 1 is one member of a family of proteins, which includes Hexokinase 2, Hexokinase 3, Glucokinase (Hexokinase 4), and Hexokinase Domain Containing 1. The aforementioned proteins demonstrate significant sequence homology, (e.g. using the NCBI BLAST engine, human HK1 and HK2 have 73% identities and 86% positives; NCBI Accessions BC008730.2 coding sequence and NP_000180.2, respectively) as well as share common conserved domains, including hexokinase domains_1 and _2 (pfam00349 and pfam03727, respectively), as well as the conserved multi-domain COG5026 Hexokinase [carbohydrate transport and metabolism].

Kelch-like 12 (KLHL12) is a protein involved in the ubiquitin ligase conjugation and wnt cell-signaling pathway. It contains 6 kelch repeat domains and a BTB (POZ) domain. Several Kelch-like and other proteins exist containing the aforementioned domains (e.g. see Table VI).

Due to both protein sequence similarity and the phenomena of intra- and inter-molecular epitope spreading [Vanderlugtand Miller (2002) Nat Rev Immunol2: 85-95], we fully expect that the aforementioned HK1 and KLHL12 homologs (see also Examples in Table VI) would show a similar performance with respect to the detection of disease-specific autoantibodies in Primary Biliary Cirrhosis (PBC). Furthermore, the use. of homologs may increase diagnostic sensitivity and/or specificity. In this Example, this will be evaluated.

### Autoantigen Expression

Will be performed as in Example 3 except that homologs of HK1 and KLHL12 will be expressed and used as autoantigens for detection of autoantibodies, such as those mentioned above in this Example and the examples of homologs listed in Table VI.

Dual-Tag Enzyme-Linked Immunosorbent Assay (T²-ELISA) of Autoantigens Will be performed as in Example 3.

### Results:

As in Example 3, in order to set diagnostic scoring thresholds for a given autoantigen species, the T²-ELISA assay will be run on a group of 22 normal patient sera and the cutoffs will then be set at 2 standard deviations above the mean for this normal cohort, for ˜95% statistical confidence. The use of this method at 2-3 standard deviations is common practice (e.g. [Liu, Wang, Li, Xu, Dai, Wang and Zhang (2009) Scand J Immunol 69: 57-63]). The T²-ELISA will then be run on 22 PBC patient sera (e.g. 22 AMA-negative and/or 22 AMA-positive). The autoantigen-specific cutoffs will then be used to score both the normal and PBC patients as autoantibody negative or positive. Autoantibody Unit calculations and data processing will be performed as in Example 3. Calculations of diagnostic sensitivity and specificity for each autoantigen species will then be performed as in Example 3.

Due to both protein sequence similarity and the phenomena of intra- and inter-molecular epitope spreading [Vanderlugt and Miller (2002) Nat Rev Immunol 2: 85-95], the expectation is that at least some of the HK1 and KLHL12 homologs will show similar diagnostic performance as in Example 3 for AMA-positive and Example 4 for AMA-negative PBC where human HK1 and KLHL12 themselves were used. It is also expected that some may perform better, either in diagnostic sensitivity or specificity, or both.

### DESCRIPTION OF THE FIGURES

FIG. 1: ELISA Based Pre-Validation of the PBC Autoantigen Hexokinase 1 (HK1) on Positive and Negative Serum Samples Randomly Selected from the Microarray Analyses. The graphed data are from the ELISA. The “+” and “−” denote if a given serum was positive or negative for HK1 autoantibodies based on either the

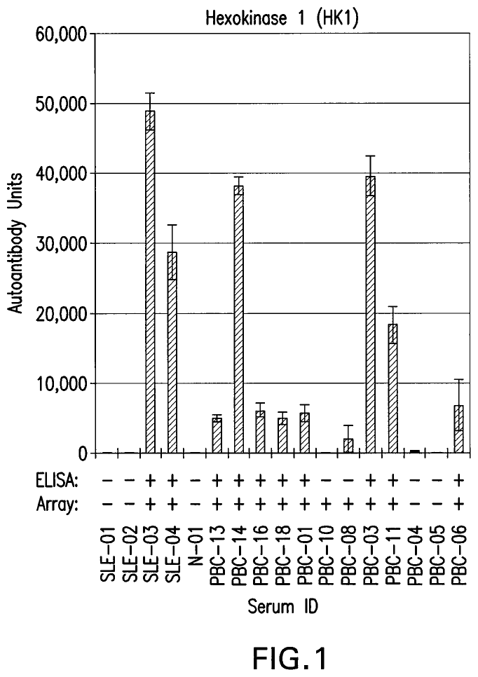

“ELISA” assay or microarray (“Array”) analyses. Serum samples prefixed with “N” are from healthy individuals, “PBC” from primary biliary cirrhosis patients, and “SLE” from systemic lupus erythematosus patients. Calculation of Autoantibody Units from the ELISA assay is detailed in Example 2.

FIG. 2: ELISA Based Pre-Validation of the PBC Autoantigen Kelch-Like 12 (KLHL12) on Positive and Negative Serum Samples Randomly Selected from the Microarray Analyses. The graphed data are from the ELISA. The “+” and “−” denote if a given serum was positive or negative for KLHL12 autoantibodies based on either the “ELISA” assay or microarray (“Array”) analyses. Serum samples prefixed with “N” are from healthy individuals and “PBC” from primary biliary cirrhosis patients. Calculation of Autoantibody Units from the ELISA assay is detailed in Example 2.

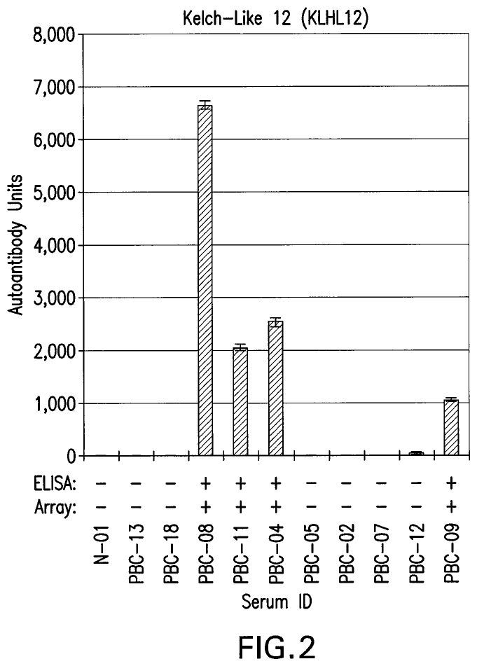

FIG. 3: ELISA Based Validation of the PBC Autoantigen Hexokinase 1 (HK1) on a new PBC Patient Cohort Never Before Tested on the Proteome Microarrays. The graphed data are the Log ₂ transformed Autoantibody Units from the ELISA analysis. Calculation of Autoantibody Units from the ELISA assay is detailed in Example 2. Patient samples were scored as HK1 negative or positive based on the cutoff values (dotted red line) which were calculated as detailed in Example 3. The red boxed region indicates the PBC cohort and the unboxed region the normal cohort.

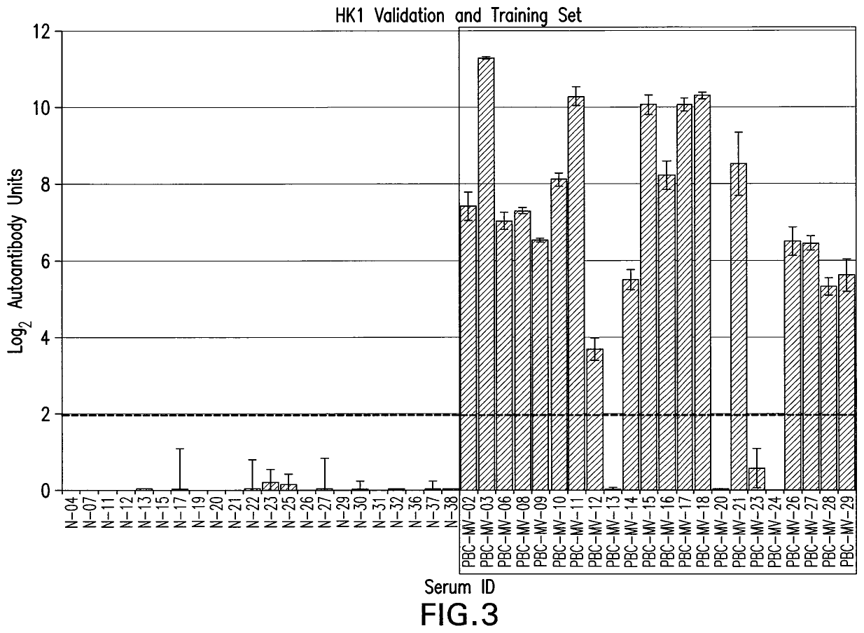

FIG. 4: ELISA Based Validation of the PBC Autoantigen Kelch-Like 12 (KLHL12) on a New PBC Patient Cohort Never Before Tested on the Proteome Microarrays. The graphed data are the Log ₂ transformed Autoantibody Units from the ELISA analysis. Calculation of Autoantibody Units from the ELISA assay is detailed in Example 2. Patient samples were scored as KLHL12 negative or positive based on the cutoff values (dotted red line) which were calculated as detailed in Example 3. The red boxed region indicates the PBC cohort and the unboxed region the normal cohort.

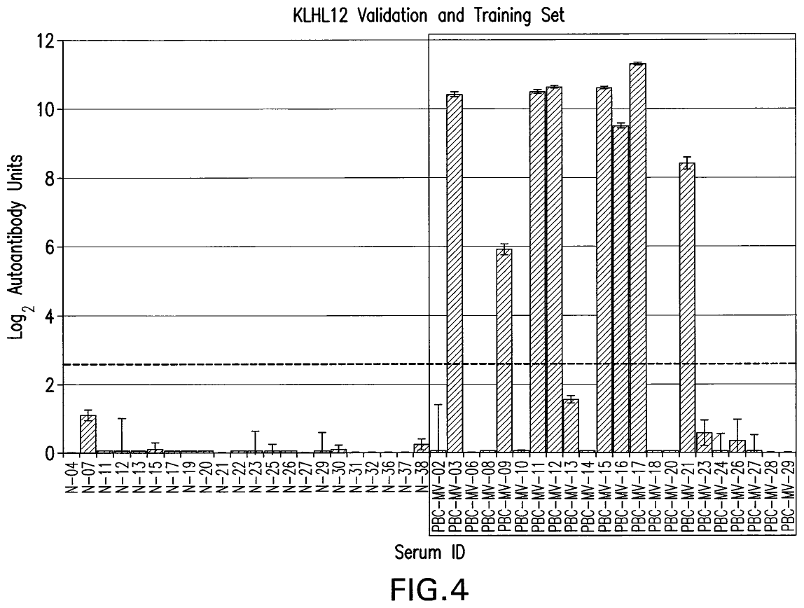

FIG. 5: Detection of the PBC Autoantigen Hexokinase 1 (HK1) on a New PBC Antimitochondrial Antibody (AMA)-Negative Cohort. The graphed data are the Log ₂ transformed Autoantibody Units from the ELISA assay, as calculated in Example 2. Dotted red line indicates the diagnostic scoring threshold, as previously determined in Example 3. HK1 detected 4 of 17 AMA-negative PBC patients (24% sensitivity). Of note, one AMA-negative PBC patient (green bar) was detected by HK1 but undetected by any of the commercially available FDA-approved ELISA assays from INOVA Diagnostics for PBC.

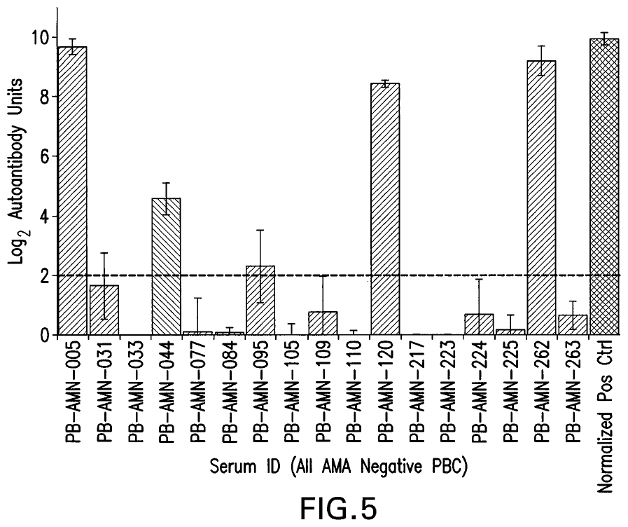

FIG. 6: Detection of the PBC Autoantigen Kelch-like 12 (KLHL12) on a New PBC Antimitochondrial Antibody (AMA)-Negative Cohort. The graphed data are the Loge transformed Autoantibody Units from the ELISA assay, as calculated in Example 2. Dotted red line indicates the diagnostic scoring threshold, as previously determined in Example 3. KLHL12 detected 6 of 17 AMA-negative PBC patients (35% sensitivity). Of note, one AMA-negative PBC patient (green bar) was detected by KLHL12 but undetected by any of the commercially available FDA-approved ELISA assays from INOVA Diagnostics for PBC.

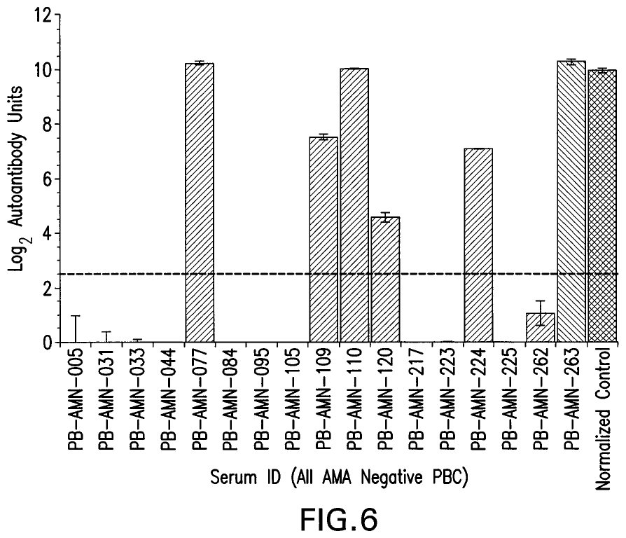

FIG. 7: Venn Diagram—Novel PBC-Specific Autoantigens, HK1 and KLHL12, Capture Previously Undetectable AMA-Negative PBC Patients. Each number represents a patient.

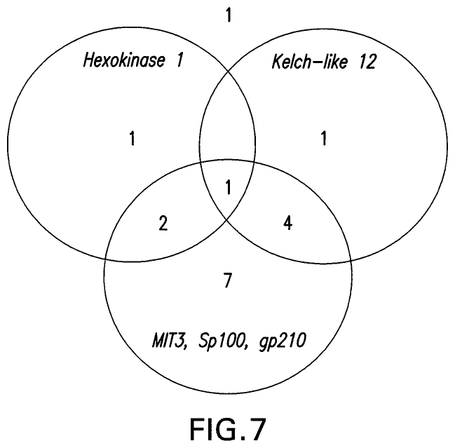

FIG. 8: Detection of Hexokinase 1 (HK1)and Kelch-like 12 (KLHL12), in Addition to INOVA Diagnostic's MIT3 Assay, May Reveal a Large Number of Previously Undiagnosed PBC Patients With Atypical Indirect Immunofluorescence Staining (IIF). Serum samples prefixed with “Cyto” or “NM” are from patients with diffuse cytoplasmic or nuclear membrane IIF staining, respectively. To avoid scale effects, graphed data for each antigen is normalized as a percent of the patient having the maximum autoantibody units for that antigen (that patient is marked with a blue arrow for each antigen). We set the Y-axis to INOVA's MIT3 cut-off of 25 units, which corresponded to 17%. All bars shown in the graph represent positive results and the lack of a bar a negative result. The “High Positive” is a selected positive control serum for each of the autoantigens.

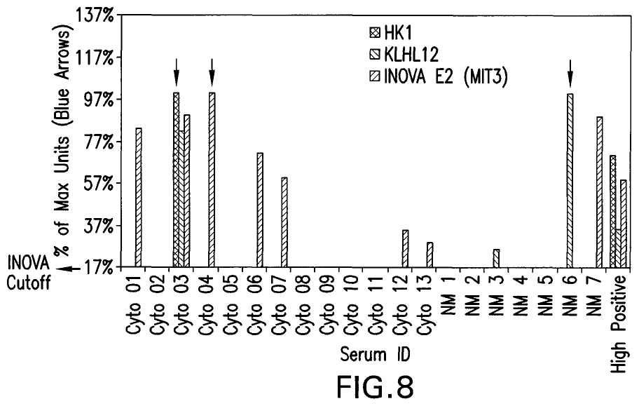

FIG. 9A: HK1 Detection By Colorimetric ELISA in Selected PBC Patients—Concordance with Chemiluminescence ELISA Readout. Colorimetric ELISA results are plotted as the signal minus background, with the background being the same serum run against an expression blank (no expressed autoantigen). The chemiluminescence ELISA score is indicated below the X-Axis by a “+” (positive) or “−” (negative). The scores for the chemiluminescent ELISA were those as already determined in Example 4 for the same sera. The bar with the green outline corresponds to the same sample from Example 4 (PB-AMN-044) to score negative on all available PBC ELISA assays from INOVA Diagnostics but positive for HK1.

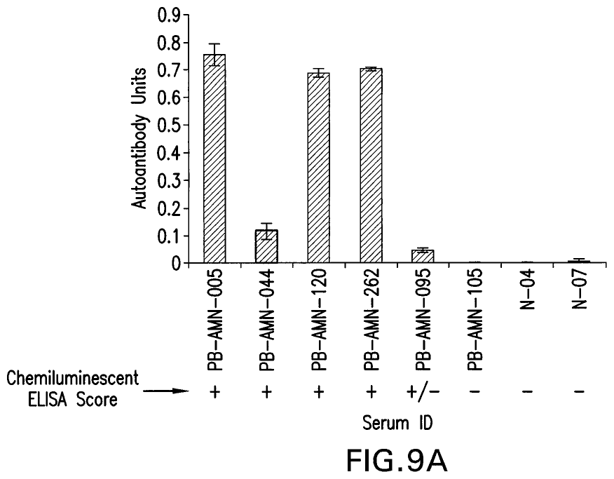

FIG. 9B: KLHL12 Detection By Colorimetric ELISA in Selected PBC Patients—Concordance with Chemiluminescence ELISA Readout. Colorimetric ELISA results are plotted as the signal minus background, with the background being the same serum run against an expression blank (no expressed autoantigen). The chemiluminescence ELISA score is indicated below the X-Axis by a “+” (positive) or “−” (negative). The scores for the chemiluminescent ELISA were those as already determined in Example 4 for the same sera. The bar with the green outline corresponds to the same sample from Example 4 (PB-AMN-263) to score negative on all available PBC ELISA assays from INOVA Diagnostics but positive for KLHL12.

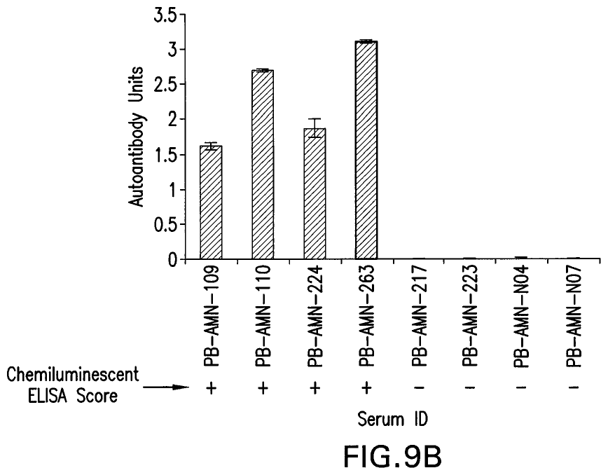

FIG. 10: Colorimetric Dot Blot of PBC Autoantigen HK1 Probed with PBC and Normal Patient Sera. Newly discovered PBC Autoantigen HK1 was spotted onto nitrocellulose, as well as buffer (negative control) and human IgG (positive control). Diluted sera from a PBC patient and normal patient was allowed to bind and washed before adding colloidal gold labeled anti-human IgG. “hIgG” is human IgG positive control; “AAg” is new PBC autoantigen HK1; “Ctrl” is negative control (carrier buffer).

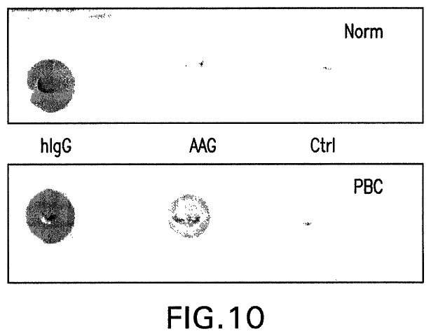

FIG. 11: Comparison of T²-ELISA to a Commercial (INOVA Diagnostics) ELISA Using the Sp100 Autoantigen and PBC Sera. Serum samples prefixed with “PBC” are from primary biliary cirrhosis patients. Red boxed region represents INOVA ELISA results; yellow boxed region represents T²-ELISA results. *Units above the “Low Positive” control (red line) are scored as diagnostically positive.

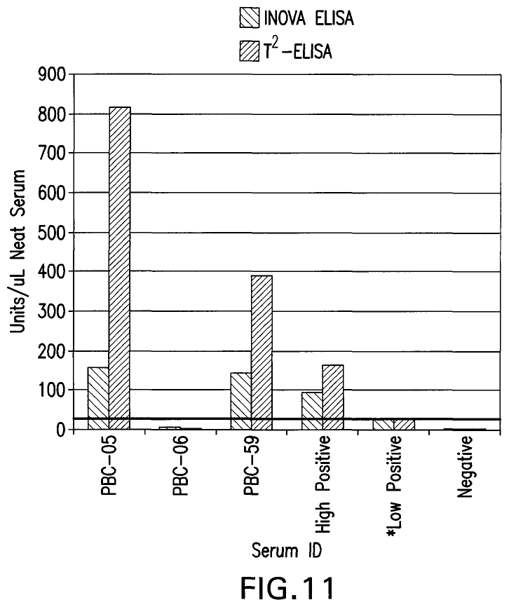

FIG. 12: T²-ELISA Versus Conventional ELISA for p53 Autoantibody Detection Cancer Sera. Normal sera are prefixed with an “N” (green box) and all others are CRC sera. Data are normalized as a percent of the maximum sera for that assay.

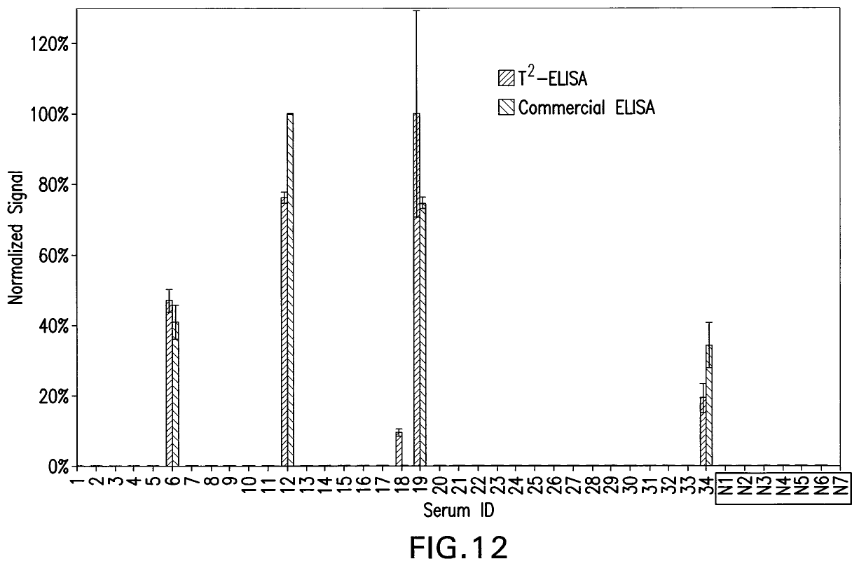

FIG. 13: Dual-Reporter and Single-Reporter T²-ELISA Assays Against Various Serum-Antigen Pairs. The graphed data are the Autoantibody Units from the ELISA analysis. Calculation of Autoantibody Units from the ELISA assay is detailed in Example 12. Blue text denotes the antigen. Serum samples prefixed with “N” are normal (from healthy individuals), “SLE” systemic lupus erythematosus and “PBC” primary biliary cirrhosis.

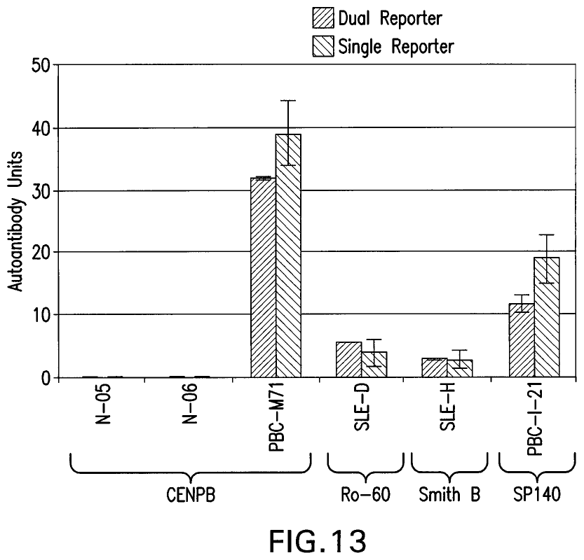

FIG. 14: Autoantibody Detection in ELISA with Pre-Purified Human Hexokinase 1 Autoantigen (HK1) Coated Directly to Polystyrene Microtiter Plate Surface. Pre-purified expressed recombinant protein autoantigen was bound directly to the polystyrene microtiter ELISA plate surface and used to assay patient serum for the presence of autoantibodies. The expected result, based on previous microarray and T²-ELISA data (Examples 1 and 2), is listed below the X-Axis as “+” (autoantibody positive) or “−” (autoantibody negative). The actual result of the assay in this Example, is shown based on the scoring cutoff in the bar graph (red dotted line), which was calculated as 2 standard deviations above the mean for the 4 expected negative samples.

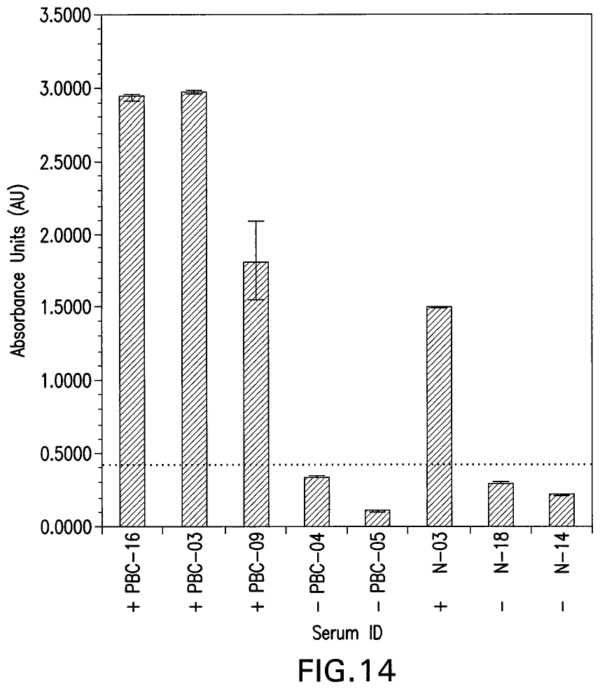

FIG. 15: Autoantibody Detection in ELISA with Pre-Purified Human Kelch-Like 12 Autoantigen (KLHL12) Coated Directly to Polystyrene Microtiter Plate Surface. Pre-purified expressed recombinant protein autoantigen was bound directly to the polystyrene microtiter ELISA plate surface and used to assay patient serum for the presence of autoantibodies. The expected result, based on previous microarray and T²-ELISA data (Examples 1 and 2), is listed below the X-Axis as “+” (autoantibody positive) or “−” (autoantibody negative). The actual result of the assay in this Example, is shown based on the scoring cutoff in the bar graph (red dotted line), which was calculated as 2 standard deviations above the mean for the 4 expected negative samples.

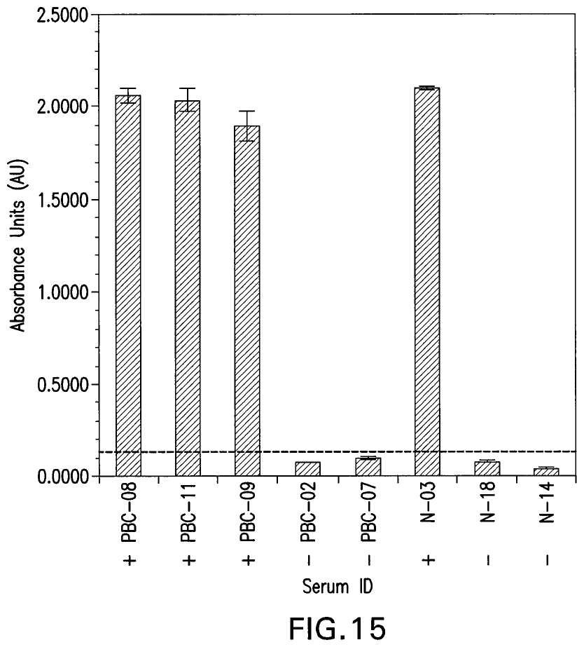

FIG. 16: Quantile Normalized Proteome Microarray (ProtoArray) Autoantibody Data for Human Hexokinase 1 (HK1) for 92 Distinct Serum Samples. Autoantibody fluorescence signal intensity, “Array Signal” (quantile normalized across the entire 92-member microarray set on a per lots basis), for each of the patient serum samples is shown for the novel autoantigen human HK1. PBC=Primary Biliary Cirrhosis; Normal or Norm=Healthy Individuals; SLE=Systemic Lupus Erythematosus; SjS=Sjogren's Syndrome; CRC=Colorectal Cancer; AIH=Autoimmune Hepatitis.

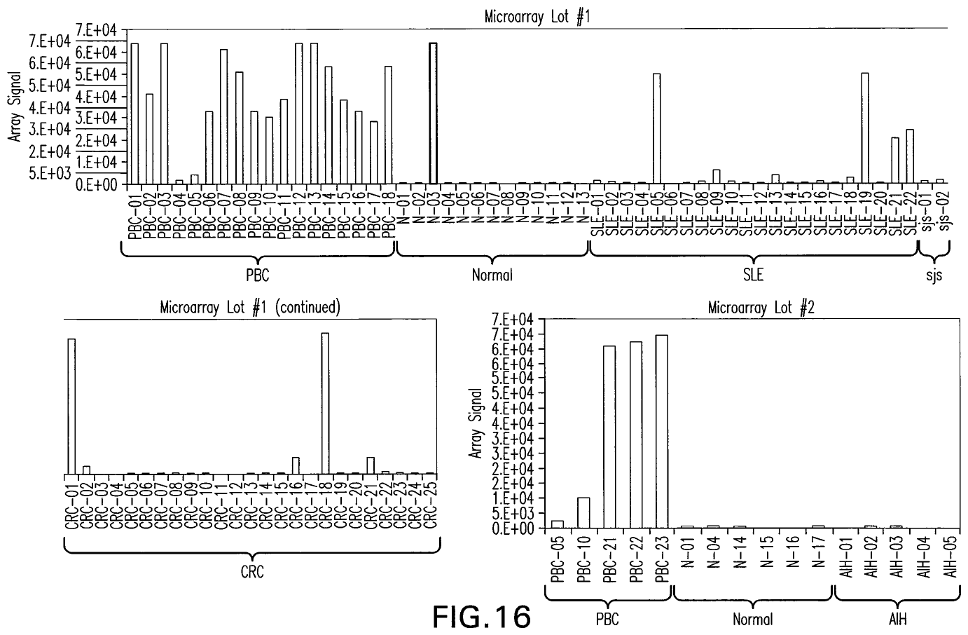

FIG. 17: Quantile Normalized Proteome Microarray (ProtoArray) Autoantibody Data for Human Kelch-Like 12 (KLHL12) for 92 Distinct Serum Samples. Autoantibody fluorescence signal intensity, “Array Signal” (quantile normalized across the entire 92-member microarray set on a per lots basis), for each of the patient serum samples is shown for the novel autoantigen human KLHL12. PBC=Primary Biliary Cirrhosis; Normal or Norm=Healthy Individuals; SLE=Systemic Lupus Erythematosus; SjS=Sjogren's Syndrome; CRC=Colorectal Cancer; AIH Autoimmune Hepatitis.

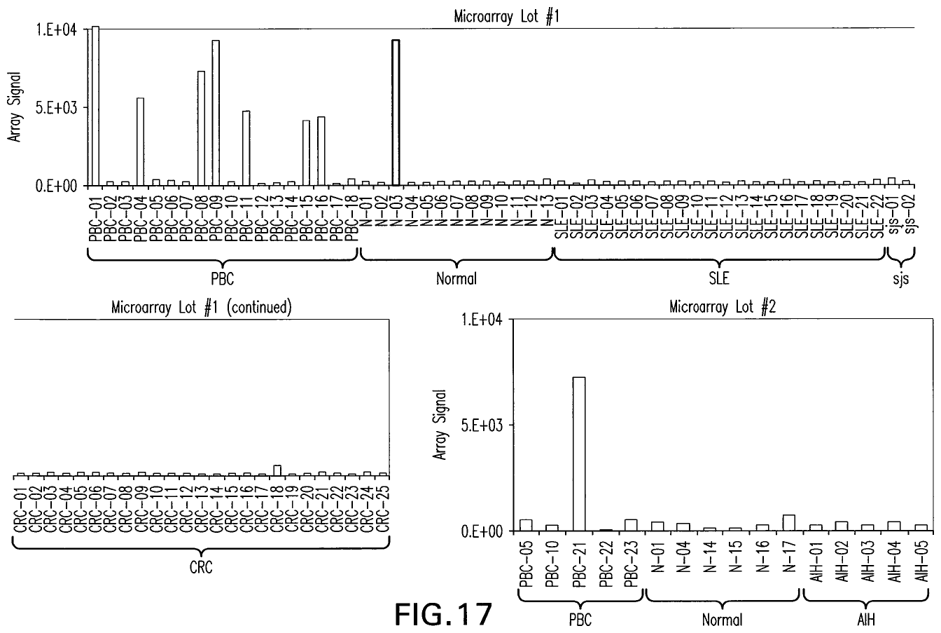

TABLE I
Primary Biliary Cirrhosis (PBC) Autoantigens

| NCBI GenBank | | | | |
|---|---|---|---|---|
| Accession (ID of | | | | Diagnostic Sensitivity |
| Nucleic Acid | | | M-Statistics | (S) & Specificity (P) in |
| Coding for Protein) | Gene | Fasta Header/Description | P-Value | PBC vs. all Non-PBC |
| BC008730.2 | HK1 | >gi\|33869444\|gb\|BC008730.2\| | 0.00000000012 | By Hit Calling Method: |
| | | Homo sapiens | | (S) 89% |
| | | hexokinase 1, transcript | | (P) 84% |
| | | variant 1, mRNA (cDNA | | By M-Statistics Method: |
| | | clone MGC: 1724 | | (S) 85% |
| | | IMAGE: 3163058), | | (P) 90% |
| | | complete cds | | |
| NM_021633.2 | KLHL12 | >gi\|21361889\|ref\| | 0.000076 | By Hit Calling Method: |
| | | NM_021633.2\| | | (S) 33% |
| | | Homo sapiens kelch- | | (P) 98% |
| | | like 12 (Drosophila) | | By M-Statistics Method: |
| | | (KLHL12), mRNA | | (S) 40% |
| | | | | (P) 97% |

TABLE II
Compiled ELISA Results for PBC-Specific Autoantigens
on Antimitochondrial Antibody (AMA)-Negative PBC Sera
PBC-Positive AMA-Negative Sera

| | INOVA | | | | | |
|---|---|---|---|---|---|---|
| | | | | PBC | | |
| | | | | Screen | AmberGen | |
| Sera ID | MIT3 | Gp210 | Sp100 | IgG/IgA | HK1 | KLHL12 |
| PB-AMN-005 | − | − | + | + | + | − |
| PB-AMN-031 | + | − | + | + | − | − |
| PB-AMN-033 | + | − | E | + | − | − |
| *PB-AMN-044 | − | − | − | − | + | − |
| PB-AMN-077 | − | − | + | E | − | + |
| ^{Δ}PB-AMN-084 | − | − | − | − | − | − |
| PB-AMN-095 | − | + | − | + | E | − |
| PB-AMN-105 | − | + | − | − | − | − |
| PB-AMN-109 | − | − | + | + | − | + |
| PB-AMN-110 | + | − | + | + | − | + |
| PB-AMN-120 | + | + | − | + | + | + |
| PB-AMN-217 | − | − | + | + | − | − |
| PB-AMN-223 | − | − | + | + | − | − |
| PB-AMN-224 | − | − | + | + | − | + |
| PB-AMN-225 | − | − | + | + | − | − |
| PB-AMN-262 | − | − | + | + | + | − |
| *PB-AMN-263 | − | − | − | − | − | + |

*Negative by all 4 INOVA tests but detected by AmberGen
^{Δ}Negative by INOVA and AmberGen tests
E Equivocal-presence or absence of autoantibodies unable to be determined

TABLE III
ELISA Scores for PBC Patient Sera.

| | Sp100 | | Sp140 |
|---|---|---|---|
| Serum ID | INOVA Score | T²-ELISA Score | T²-ELISA Score |
| PB-AMP-002 | + | + | + |
| PB-AMN-005 | + | + | − |
| PB-AMP-006 | − | − | − |
| PB-AMP-011 | − | − | − |
| PB-AMP-018 | + | + | − |
| *PB-AMP-020 | − | − | + |
| PB-AMP-021 | + | + | + |
| PB-AMP-024 | + | + | − |
| PB-AMP-029 | + | + | − |
| PB-AMN-031 | + | + | − |
| PB-AMN-033 | E | − | − |
| PB-AMP-035 | + | + | − |
| PB-AMP-036 | + | + | − |
| PB-AMP-039 | + | + | + |
| PB-AMP-046 | − | − | − |
| PB-AMP-047 | + | + | + |
| PB-AMP-048 | + | + | − |
| PB-AMP-059 | + | + | − |
| PB-AMP-063 | + | + | − |
| PB-AMP-066 | + | + | − |
| PB-AMP-068 | E | − | − |
| PB-AMN-077 | + | + | + |
| PB-AMP-080 | + | + | − |
| *PB-AMN-084 | − | − | + |
| PB-AMP-102 | + | + | − |
| PB-AMN-109 | + | + | + |
| PB-AMN-110 | + | + | − |
| PB-AMP-113 | + | + | − |
| PB-AMP-122 | + | + | + |
| PB-AMN-217 | + | + | + |
| PB-AMP-218 | + | + | − |
| PB-AMN-223 | + | + | + |
| PB-AMN-224 | + | + | − |
| PB-AMN-225 | + | + | − |
| PB-AMN-262 | + | + | + |

E = equivocal, i.e. inconclusive (too close to cutoff; only used in INOVA assay).
Asterisks indicate samples negative for Sp100 but positive for Sp140.

TABLE IV
Dual detection ELISA is as efficient as single detection ELISA

| Reporter Labeled | AP Detection | HRP Detection (Rap55 |
|---|---|---|
| Probes Added | (Autoantibody) | Autoantigen Expression) |
| A. Single Reporter (Control) Versus Dual Reporter | | |
| (Percent of Control) T²-ELISA Against Rap55 | | |
| Autoantigen and PBC Patient Serum | | |
| Percent of Control | | |
| anti-VSV-HRP | 0.02 | 100.00 (control) |
| anti-human-AP | 100.00 (control) | 0.23 |
| anti-VSV-AP, then | 97.38 | 96.48 |
| anti-human AP | | |
| B. Single Reporter Versus Dual Reporter T²-ELISA Against | | |
| Rap55 Autoantigen and PBC Patient Serum (Signal to Noise) | | |
| Signal to Noise | | |
| anti-VSV-HRP | 3.98 | 697.86 (control) |
| anti-human-AP | 20.15 (control) | 4.51 |
| anti-VSV-AP, then | 20.22 | 760.00 |
| anti-human AP | | |

TABLE V
Human Hexokinase 1 (HK1) and Human Kelch-Like 12
(KLHL12) Sequences on ProtoArray v.4.0 (Invitrogen, Carlsbad, CA)
(Example 1), on the T²-ELISA (several Examples) and on the
Conventional ELISA of Example 12.

| | | |
|---|---|---|
| NCBI | | |
| GenBank or | | |
| Protein | Fasta Header/ | |
| Accession | Description | Sequence |

ProtoArray v.4.0 (Invitrogen, Carlsbad, CA)- Recombinant human HK1 and
KLHL12 expressed in a baculovirus/Sf9 insect cell system. Note that HK1 and
KLHL12 from the ProtoArray contained an N-terminal GST fusion tag (sequence
not shown) commonly known to those skilled in the art.

| | | |
|---|---|---|
| BC008730.2 | >gi\|33869444\|gb\|BC008730.2\| | MIAAQLLAYYFTELKDDQVKKIBKYLYAMRLSDETLIDIMTRFRKEMKNGLSRDFNPTA |
| SEQ ID NO: 1 | Homo sapiens hexokinase 1, | TVKMLPTFVRSIPDGSEKGDFIALDLGGSSFRILRVQVNHEKNQNVHMESEVYDTPENI |
| | transcript variant 1, mRNA | VHGSGSQLFDHVAECLGDFMEKRKIKDKKLPVGFTFSFPCQQSKIDEAILITWTKRFKA |
| | (cDNA clone MGC:1724 | SGVEGADVVKLLNKAIKKRGDYDANIVAVVNDTVGTMMTCGYDDQHCEVGLIIGTGTNA |
| | IMAGE:3163058), complete | CYMEELRHIDLVEGDEGRMCINTEWGAFGDDGSLEDIRTEFDREIDRGSLNPGKQLFEK |
| | cds | MVSGMYLGELVRLILVKMAKEGLLFEGRITPELLTRGKFNTSDVSAIEKNKEGLHNAKE |
| | | ILTRLGVEPSDDDCVSVQHVCTIVSFRSANLVAATLGAILNRLRDNKGTPRLRTTVGVD |
| | | GSLYKTHPQYSRRFHKTLRRLVPDSDVRFLLSESGSGKGAAMVTAVAYRLAEQHRQIEE |
| | | TLAHFHLTKDMLLEVKKRMRAEMELGLRKQTHNNAVVKMLPSFVRRTPDGTENGDFLAL |
| | | DLGGTNFRVLLVKIRSGKKRTVEMHNKIYAIPIEIMQGTGEELFDHIVSCISDFLDYMG |
| | | IKGPRMPLGFTFSFPCQQTSLDAGILITWTKGFKATDCVGHDVVTLLRDAIKRREEFDL |
| | | DVVAVVNDTVGTMMTCAYEEPTCEVGLIVGTGSNACYMEEMKNVEMVEGDQGQMCINME |
| | | WGAFGDNGCLDDIRTHYDRLVDEYSLNAGKQRYEKMISGMYLGEIVRNILIDFTKKGFL |
| | | FRGQISETLKTRGIFETKFLSQIESDRLALLQVRAILQQLGLNSTCDDSILVKTVCGVV |
| | | SRRAAQLCGAGMAAVVDKIRENRGLDRLNVTVGVDGTLYKLHPHFSRIMHQTVKELSPK |
| | | CNVSFLLSEDGSGKGAALITAVGVRLRTEASS |
| NM_021633.2 | >gi\|21361889\|ref\|NM_021633.2\| | MGGIMAPKDIMTNTHAKSILNSMNSLRKSNTLCDVTLRVEQKDFPAHRIVLAACSDYFC |
| SEQ ID NO: 2 | Homo sapiens kelch-like 12 | AMFTSELSEKGKPYVDIQGLTASTMEILLDFVYTETVHVTVENVQELLPAACLLQLKGV |
| | (Drosophila)(KLHL12), mRNA | KQACCEFLESQLDPSNCLGIRDFAETHNCVDLMQAAEVFSQKHFPEVVQHEEFILLSQG |
| | | EVEKLIKCDEIQVDSEEPVFEAVINWVKHAKKEREESLPNLLQYVRMPLLTPRYITDVI |
| | | DAEPFIRCSLQCRDLVDEAKKFHLRPELRSQMQGPRTRARLGANEVLLVVGGFGSQQSP |
| | | IDVVEKYDPKTQEWSFLPSITRKRRYVASVSLHDRIYVIGGYDGRSRLSSVECLDYTAD |
| | | EDGVWYSVAPMNVRRGLAGATTLGDMIYVSGGFDGSRRHTSMERYDPNIDQWSMLGDMQ |
| | | TAREGAGLVVASGVIYCLGGYDGLNILNSVEKYDPHTGHWTNVTPMATKRSGAGVALLN |
| | | DHIYVVGGFDGTAHLSSVEAYNIRTDSWTTVTSMTTPRCYVGATVLRGRLYAIAGYDGN |
| | | SLLSSIECYDPIIDSWEVVTSMGTQRCBAGVCVLREK |

T²-ELISA - Recombinant human HK1 and KLHL12 cell-free expressed in a rabbit
reticulocyte lysate. Note that the underlined sequences are exogenously added
N-terminal and C-terminal epitope tags as well as vector-derived sequences.

| | | |
|---|---|---|
| CV026580 | >gi\|51484591\|gb\|CV026580.1\| | MAIYTDIEMNRLGKMIAAQLLAYYFTELKDDQVKKIDKYLYAMRLSDETLIDIMTRFRK |
| (EST) | CV026580 4566 Full Length | EMKNGLSRDFNPTATVKMLPTFVRSIPDGSEKGDFIALDLGGSSFRILRVQVNHEKNQN |
| SEQ ID NO: 3 | cDNA from the Mammalian Gene | VHMESEVYDTPENIVHGSGSQLFDHVAECLGDFMEKRKIKDKKLPVGFTFSFPCQQSKI |
| | Collection Homo sapiens cDNA | DEAILITWTKRFKASGVEGADVVKLLNKAIKKRGDYDANIVAVVNDTVGTMMTCGYDDQ |
| | 5′ similar to BC008730 | HCEVGLIIGTGTNACYMEELRHIDLVEGDEGRMCINTEWGAFGDDGSLEDIRTEFDREI |
| | (HK1), mRNA sequence | DRGSLNPGKQLFEKMVSGMYLGELVRLILVKMAKEGLLFEGRITPELLTRGKFNTSDVS |
| | | AIEKNKEGLHNAKEILTRLGVEPSDDDCVSVQHVCTIVSFRSANLVAATLGAILNRLRD |
| | | NKGTPRLRTTVGVDGSLYKTHPQYSRRFHKTLRRLVPDSDVRFLLSESGSGKGAAMVTA |
| | | VAYRLAEQHRQIEETLAHFHLTKDMLLEVKKRMRAEMELGLRKQTHNNAVVKMLPSFVR |
| | | RTPDGTENGDFLALDLGGTNFRVLLVKIRSGKKRTVEMHNKIYAIPIEIMQGTGEELFD |
| | | HIVSCISDFLDYMGIKGPRMPLGFTFSFPCQQTSLDAGILITWTKGFKATDCVGHDVVT |
| | | LLRDAIKRREEFDLDVVAVVNDTVGTMMTCAYEEPTCEVGLIVGTGSNACYMEEMKNVE |
| | | MVEGDQGQMCINMEWGAFGDNGCLDDIRTHYDRLVDEYSLNAGKQRYEKMISGMYLGEI |
| | | VRNILIDFTKKGFLFRGQISETLKTRGIFETKFLSQIESDRLALLQVRAILQQLGLNST |
| | | CDDSILVKTVCGVVSRRAAQLCGAGMAAVVDKIRENRGLDRLNVTVGVDGTLYKLHPHF |
| | | SRIMHQTVKELSPKCNVSFLLSEDGSGKGAALITAVGVRLRTEASSLSRELVDPNSVQA |
| | | RLQDVDGTIDTRSKLAAAQLYTRASQPELAPEDPEDLEHHHHHH |
| BC003183.1 | >gi\|13112018\|gb\|BC003183.1\| | MYTDIEMNRLGKMGGIMAPKDIMrNTHAKSILNSMNSLRKSNTLCDVTLRVEQKDFPAH |
| SEQ ID NO: 4 | Homo sapiens kelch-like | RIVLAACSDYFCAMFTSELSEKGKPYVDIQGLTASTMEILLDFVYTETVHVTVENVQEL |
| | 12 (Drosophila), mRNA | LPAACLLQLKGVKQACCEFLESQLDPSNCLGIRDFAETHNCVDLMQAAEVFSQKHFPEV |
| | (cDNA clone MGC:4435 | VQHEEFILLSQGEVEKLIKCDEIQVDSEEPVFEAVINWVKHAKKEREESLPNLLQYVRM |
| | IMAGE:2958852), complete | PLLTPRYITDVIDAEPFIRCSLQCRDLVDEAKKFHLRPELRSQMQGPRTRARLGANEVL |
| | cds | LVVGGFGSQQSPIDVVEKYDPKTQEWSFLPSITRKRRYVASVSLHDRIYVIGGYDGRSR |
| | | LSSVECLDYTADEDGVWYSVAPMNVRRGLAGATTLGDMIYVSGGFDGSRRHTSMERYDP |
| | | NIDQWSMLGDMQTAREGAGLVVASGVIYCLGGYDGLNILNSVEKYDPHTGHWTNVTPMA |
| | | TKRSGAGVALLNDHIYVVGGFDGTAHLSSVEAYNIRTDSWTTVTSMTTPRCYVGATVLR |
| | | GRLYAIAGYDGNSLLSSIECYDPIIDSWEVVTSMGTQRCDAGVCVLREKQPELAPEDPE |
| | | D |

Conventional ELISA- Recombinant human HK1 and KLHL12 (Abnova, Taipei
City, 114, Taiwan) cell-free expressed in a wheat germ based system. Note that
HK1 and KLHL12 contained an N-terminal GST fusion tag (sequence not shown)
commonly known to those skilled in the art.

| | | |
|---|---|---|
| AAH08730 | >gi\|14250554\|gb\|AAH08730.1\| | MIAAQLLAYYFTELKDDQVKKIDKYLYAMRLSDETLIDIMTRFRKEMKNGLSRDFNPTA |
| SEQ ID NO: 5 | Hexokinase 1 [Homo sapiens] | TVKMLPTFVRSIPDGSEKGDFIALDLGGSSFRILRVQVNHEKNQNVHMESEVYDTPENI |
| | | VHGSGSQLFDHVAECLGDFMEKRKIKDKKLPVGFTFSFPCQQSKIDEAILITWTKRFKA |
| | | SGVEGADVVKLLNKAIKKRGDYDANIVAVVNDTVGTMMTCGYDDQHCEVGLIIGTGTNA |
| | | CYMEELRHIDLVEGDEGRMCINTEWGAFGDDGSLEDIRTEFDREIDRGSLNPGKQLFEK |
| | | MVSGMYLGELVRLILVKMAKEGLLFEGRITPELLTRGKFNTSDVSAIEKNKEGLHNAKE |
| | | ILTRLGVEPSDDDCVSVQHVCTIVSFRSANLVAATLGAILNRLRDNKGTPRLRTTVGVD |
| | | GSLYKTHPQYSRRFHKTLRRLVPDSDVRFLLSESGSGKGAAMVTAVAYRLAEQHRQIEE |
| | | TLAHFHLTKDMLLEVKKRMRAEMELGLRKQTHNNAVVKMLPSFVRRTPDGTENGDFLAL |
| | | DLGGTNFRVLLVKIRSGKKRTVEMHNKIYAIPIEIMQGTGEELFDHIVSCISDFLDYMG |
| | | IKGPRMPLGFTFSFPCQQTSLDAGILITWTKGFKATDCVGHDVVTLLRDAIKRREEFDL |
| | | DVVAVVNDTVGTMMTCAYEEPTCEVGLIVGTGSNACYMEEMKNVEMVEGDQGQMCINME |
| | | WGAFGDNGCLDDIRTHYDRLVDEYSLNAGKQRYEKMISGMYLGEIVRNILIDFTKKGFL |
| | | FRGQISETLKTRGIFETKFLSQIESDRLALLQVRAILQQLGLNSTCDDSILVKTVCGVV |
| | | SRRAAQLCGAGMAAVVDKIRENRGLDRLNVTVGVDGTLYKLHPHFSRIMHQTVKELSPK |
| | | CNVSFLLSEDGSGKGAALITAVGVRLRTEASS |
| NP_067646.1 | >gi\|11056006\|ref\| | MGGIMAPKDIMTNTHAKSILNSMNSLRKSNTLCDVTLRVEQKDFPAHRIVLAACSDYFC |
| SEQ ID NO: 6 | NP_067646.1\|kelch-like 12 | AMFTSELSEKGKPYVDIQGLTASTMEILLDFVYTETVHVTVENVQELLPAACLLQLKGV |
| | [Homo sapiens] | KQACCEFLESQLDPSNCLGIRDFAETHNCVDLMQAAEVFSQKHFPEVVQHEEFILLSQG |
| | | EVEKLIKCDEIQVDSEEPVFEAVINWVKHAKKEREESLPNLLQYVRMPLLTPRYITDVI |
| | | DAEPFIRCSLQCRDLVDEAKKFHLRPELRSQMQGPRTRARLGANEVLLVVGGFGSQQSP |
| | | IDVVEKYDPKTQEWSFLPSITRKRRYVASVSLHDRIYVIGGYDGRSRLSSVECLDYTAD |
| | | EDGVWYSVAPMNVRRGLAGATTLGDMIYVSGGFDGSRRHTSMERYDPNIDQWSMLGDMQ |
| | | TAREGAGLVVASGVIYCLGGYDGLNILNSVEKYDPHTGHWTNVTPMATKRSGAGVALLN |
| | | DHIYVVGGFDGTAHLSSVEAYNIRTDSWTTVTSMTTPRCYVGATVLRGRLYAIAGYDGN |
| | | SLLSSIECYDPIIDSWEVVTSMGTQRCDAGVCVLREK |

TABLE VI
Examples of Homologous Sequences for HK1 and KLHL12

| | | |
|---|---|---|
| NCBI Protein | | |
| Accession | Fasta Header/ | |
| (Gene Name) | Description | Sequence |

HEXOKINASE 1 and Homologs

| | | |
|---|---|---|
| NP_277031.1 | >gi\|15991827\|ref\| | MDCEHSLSLPCRGAEAWEIGIDKYLYAMRLSDETLIDIMTRFRKEMKNGLSRDFNPTATVK |
| (HK1) | NP_277031.1\|hexokinase 1 | MLPTFVRSIPDGSEKGDFIALDLGGSSFRILRVQVNHEKNQNVHMESEVYDTPENIVHGSG |
| SEQ ID NO: 7 | isoform HKI-R | SQLFDHVAECLGDFMEKRKIKDKKLPVGFTFSFPCQQSKIDEAILITWTKRFKASGVEGAD |
| | (transcript variant 2) | VVKLLNKAIKKRGDYDANIVAVVNDTVGTMMTCGYDDQHCEVGLIIGTGTNACYMEELRHI |
| | [Homo sapiens] | DLVEGDEGRMCINTEWGAFGDDGSLEDIRTEFDREIDRGSLNPGKQLFEKMVSGMYLGELV |
| | | RLILVKMAKEGLLFEGRITPELLTRGKFNTSDVSAIEKNKEGLHNAKEILTRLGVEPSDDD |
| | | CVSVQHVCTIVSFRSANLVAATLGAILNRLRDNKGTPRLRTTVGVDGSLYKTHPQYSRRFH |
| | | KTLRRLVPDSDVRFLLSESGSGKGAAMVTAVAYRLAEQHRQIEETLAHFHLTKDMLLEVKK |
| | | RMRAEMELGLRKQTHNNAVVKMLPSFVRRTPDGTENGDFLALDLGGTNFRVLLVKIRSGKK |
| | | RTVEMHNKIYAIPIEIMQGTGEELFDHIVSCISDFLDYMGIKGPRMPLGFTFSFPCQQTSL |
| | | DAGILITWTKGFKATDCVGHDVVTLLRDAIKRREEFDLDVVAVVNDTVGTMMTCAYEEPTC |
| | | EVGLIVGTGSNACYMEEMKNVEMVEGDQGQMCINMEWGAFGDNGCLDDIRTHYDRLVDEYS |
| | | LNAGKQRYEKMISGMYLGEIVRNILIDFTKKGFLFRGQISETLKTRGIFETKFLSQIESDR |
| | | LALLQVRAILQQLGLNSTCDDSILVKTVCGVVSRRAAQLCGAGMAAVVDKIRENRGLDRLN |
| | | VTVGVDGTLYKLHPHFSRIMHQTVKELSPKCNVSFLLSEDGSGKGAALITAVGVRLRTEAS |
| | | S |
| NP_000180.2 | >gi\|15553127\|ref\| | MIASHLLAYFFTELNHDQVQKVDQYLYHMRLSDETLLEISKRFRKEMEKGLGATTHPTAAV |
| (HK2) | NP_000180.2\|hexokinase 2 | KMLPTFVRSTPDGTEHGEFLALDLGGTNFRVLWVKVTDNGLQKVEMENQIYAIPEDIMRGS |
| SEQ ID NO: 8 | [Homo sapiens] | GTQLFDHIAECLANFMDKLQIKDKKLPLGFTFSFPCHQTKLDESFLVSWTKGFKSSGVEGR |
| | | DVVALIRKAIQRRGDFDIDIVAVVNDTVGTMMTCGYDDHNCEIGLIVGTGSNACYMEEMRH |
| | | IDMVEGDEGRMCINMEWGAFGDDGSLNDIRTEFDQEIDMGSLNPGKQLFEKMISGMYMGEL |
| | | VRLILVKMAKEELLFGGKLSPELLNTGRFETKDISDIEGEKDGIRKAREVLMRLGLDPTQE |
| | | DCVATHRICQIVSTRSASLCAATLAAVLQRIKENKGEERLRSTIGVDGSVYKKHPHFAKRL |
| | | HKTVRRLVPGCDVRFLRSEDGSGKGAAMVTAVAYRLADQHRARQKTLEHLQLSHDQLLEVK |
| | | RRMKVEMERGLSKETHASAPVKMLPTYVCATPDGTEKGDFLALDLGGTNFRVLLVRVRNGK |
| | | WGGVEMHNKIYAIPQEVMHGTGDELFDHIVQCIADFLEYMGMKGVSLPLGFTFSFPCQQNS |
| | | LDESILLKWTKGFKASGCEGEDVVTLLKEAIHRREEFDLDVVAVVNDTVGTMMTCGFEDPH |
| | | CEVGLIVGTGSNACYMEEMRNVELVEGEEGRMCVNMEWGAFGDNGCLDDFRTEFDVAVDEL |
| | | SLNPGKQRFEKMISGMYLGEIVRNILIDFTKRGLLFRGRISERLKTRGIFETKFLSQIESD |
| | | CLALLQVRAILQHLGLESTCDDSIIVKEVCTVVARRAAQLCGAGMAAVVDRIRENRGLDAL |
| | | KVTVGVDGTLYKLHPHFAKVMHETVKDLAPKCDVSFLQSEDGSGKGAALITAVACRIREAG |
| | | QR |
| NP_002106.2 | >gi\|194097330\|ref\| | MDSIGSSGLRQGEETLSCSEEGLPGPSDSSELVQECLQQFKVTRAQLQQIQASLLGSMEQA |
| (HK3) | NP_002106.2\|hexokinase | LRGQASPAPAVRMLPTYVGSTPHGTEQGDFVVLELGATGASLRVLWVTLTGIEGHRVEPRS |
| SEQ ID NO: 9 | 3 [Homo sapiens] | QEFVIPQEVMLGAGQQLFDFAAHCLSEFLDAQPVNKQGLQLGFSFSFPCHQTGLDRSTLIS |
| | | WTKGFRCSGVEGQDVVQLLRDAIRRQGAYNIDVVAVVNDTVGTMMGCEPGVRPCEVGLVVD |
| | | TGTNACYMEEARHVAVLDEDRGRVCVSVEWGSFSDDGALGPVLTTFDHTLDHESLNPGAQR |
| | | FEKMIGGLYLGELVRLVLAHLARCGVLFGGCTSPALLSQGSILLEHVAEMEDPSTGAARVH |
| | | AILQDLGLSPGASDVELVQHVCAAVCTRAAQLCAAALAAVLSCLQHSREQQTLQVAVATGG |
| | | RVCERHPRFCSVLQGTVMLLAPECDVSLIPSVDGGGRGVAMVTAVAARLAAHRRLLEETLA |
| | | PFRLNHDQLAAVQAQMRKAMAKGLRGEASSLRMLPTFVRATPDGSERGDFLALDLGGTNFR |
| | | VLLVRVTTGVQITSEIYSIPETVAQGSGQQLFDHIVDCIVDFQQKQGLSGQSLPLGFTFSF |
| | | PCRQLGLDQGILLNWTKGFKASDCEGQDVVSLLREAITRRQAVELNVVAIVNDTVGTMMSC |
| | | GYEDPRCEIGLIVGTGTNACYMEELRNVAGVPGDSGRMCINMEWGAFGDDGSLAMLSTRFD |
| | | ASVDQASINPGKQRFEKMISGMYLGEIVRHILLHLTSLGVLFRGQQIQRLQTRDIFKTKFL |
| | | SEIESDSLALRQVRAILEDLGLPLTSDDALMVLEVCQAVSQRAAQLCGAGVAAVVEKIREN |
| | | RGLEELAVSVGVDGTLYKLHPRFSSLVAATVRELAPRCVVTFLQSEDGSGKGAALVTAVAC |
| | | RLAQLTRV |
| NP_277042.1 | >gi\|15967159\|ref\| | MAMDVTRSQAQTALTLVEQILAEFQLQEEDLKKVMRRMQKEMDRGLRLETHEEASVKMLPT |
| (HK4) | NP_277042.1\|glucokinase | YVRSTPEGSEVGDFLSLDLGGTNFRVMLVKVGEGEEGQWSVKTKHQMYSIPEDAMTGTAEM |
| SEQ ID NO: 10 | isoform 2 [Homo sapiens] | LFDYISECISDFLDKHQMKHKKLPLGFTFSFPVRHEDIDKGILLNWTKGFKASGAEGNNVV |
| | | GLLRDAIKRRGDFEMDVVAMVNDTVATMISCYYEDHQCEVGMIVGTGCNACYMEEMQNVEL |
| | | VEGDEGRMCVNTEWGAFGDSGELDEFLLEYDRLVDESSANPGQQLYEKLIGGKYMGELVRL |
| | | VLLRLVDENLLFHGEASEQLRTRGAFETRFVSQVESDTGDRKQIYNILSTLGLRPSTTDCD |
| | | IVRRACESVSTRAAHMCSAGLAGVINRMRESRSEDVMRITVGVDGSVYKLHPSFKERFHAS |
| | | VRRLTPSCEITFIESEEGSGRGAALVSAVACKKACMLGQ |
| NP_079406.3 | >gi\|156151420\|ref\| | MFAVHLMAFTESKLKEDQIKKVDRFLYHMRLSDDTLLDIMRRFRAEMEKGLAKDTNPTAAV |
| (HKDC1) | NP_079406.3\|hexokinase | KMLPTEVRAIPDGSENGEFLSLDLGGSKERVLKVQVAEEGKRHVQMESQFYPTPNETIRGN |
| SEQ ID NO: 11 | domain containing 1 | GTELFEYVADCLADFMKTKDLKHKKLPLGLTFSFPCRQTKLEEGVLLSWTKKFKARGVQDT |
| | [Homo sapiens] | DVVSRLTKAMRRHKDMDVDILALVNDTVGTMMTCAYDDPYCEVGVIIGTGTNACYMEDMSN |
| | | IDLVEGDEGRMCINTEWGAFGDDGALEDIRTEFDRELDLGSLNPGKQLFEKMISGLYLGEL |
| | | VRLILLKMAKAGLLEGGEKSSALHTKGKIETRHVAAMEKYKEGLANTREILVDLGLEPSEA |
| | | DCIAVQHVCTIVSERSANLCAAALAAILTRLRENKKVERLRTTVGMDGTLYKIHPQYPKRL |
| | | HKVVRKLVPSCDVRELLSESGSTKGAAMVTAVASRVQAQRKQIDRVLALFQLTREQLVDVQ |
| | | AKMRAELEYGLKKKSHGLATVRMLPTYVCGLPDGTEKGKFLALDLGGTNERVLLVKIRSGR |
| | | RSVRMYNKIFATPLEIMQGTGEELFDHIVQCIADFLDYMGLKGASLPLGETESFPCRQMSI |
| | | DKGTLIGWTKGFKATDCEGEDVVDMLREAIKRRNEFDLDIVAVVNDTVGTMMTCGYEDPNC |
| | | EIGLIAGTGSNMCYMEDMRNIEMVEGGEGKMCINTEWGGEGDNGCIDDIRTRYDTEVDEGS |
| | | LNPGKQRYEKMTSGMYLGEIVRQILIDLTKQGLLFRGQISERLRTRGIFETKFLSQIESDR |
| | | LALLQVRRILQQLGLDSTCEDSIVVKEVCGAVSRRAAQLCGAGLAAIVEKRREDQGLEHLR |
| | | ITVGVDGTLYKLHPHFSRILQETVKELAPRCDVTFMLSEDGSGKGAALITAVAKRLQQAQK |
| | | EN |

KELCH-LIKE 12 and Homologs

| | | |
|---|---|---|
| NP_067646.1 | >gi\|11056006\|ref\| | MGGIMAPKDIMTNTHAKSILNSMNSLRKSNTLCDVTLRVEQKDEPAHRIVLAACSDYFCAM |
| (KLHL12) | NP_067646.1\|kelch-like 12 | FTSELSEKGKPYVDIQGLTASTMEILLDEVYTETVHVTVENVQELLPAACLLQLKGVKQAC |
| SEQ ID NO: 12 | [Homo sapiens] | CEFLESQLDPSNCLGIRDFAETHNCVDLMQAAEVESQKHEPEVVQHEEFILLSQGEVEKLI |
| | | KCDEIQVDSEEPVFEAVINWVKHAKKEREESLPNLLQYVRMPLLTPRYITDVIDAEPFIRC |
| | | SLQCRDLVDEAKKEHLRPELRSQMQGPRTRARLGANEVLLVVGGEGSQQSPIDVVEKYDPK |
| | | TQEWSFLPSITRKRRYVASVSLHDRIYVIGGYDGRSRLSSVECLDYTADEDGVWYSVAPMN |
| | | VRRGLAGATTLGDMIYVSGGEDGSRRHTSMERYDPNIDQWSMLGDMQTAREGAGLVVASGV |
| | | IYCLGGYDGLNILNSVEKYDPHTGHWTNVTPMATKRSGAGVALLNDHIYVVGGFDGTAHLS |
| | | SVEATNIRTDSWTTVTSMTTPRCYVGATVLRGRLYAIAGYDGNSLLSSIECYDPIIDSWEV |
| | | VTSMGTQRCDAGVCVLREK |
| NP_055273.2 | >gi\|40807500\|ref\| | MEGKPMRRCTNIRPGETGMDVTSRCTLGDPNKLPEGVPQPARMPYISDKHPRQTLEVINLL |
| (KLHL20) | NP_055273.2\|kelch-like 20 | RKHRELCDVVLVVGAKKIYAHRVILSACSPYFRAMFTGELAESRQTEVVIRDIDERAMELL |
| SEQ ID NO: 13 | [Homo sapiens] | IDFATTSQITVEEGNVQTLLPAACLLQLAEIQEACCEFLKRQLDPSNCLGIRAFADTHSCR |
| | | ELLRIADKFTQHNFQEVMESEEFMLLPANQLIDIISSDELNVRSEEQVFNAVMAWVKYSIQ |
| | | ERRPQLPQVLQHVRLPLLSPKFLVGTVGSDPLIKSDEECRDLVDEAKNYLLLPQERPLMQG |
| | | PRTRPRKPIRCGEVLFAVGGWCSGDAISSVERYDPQTNEWRMVASMSKRRCGVGVSVLDDL |
| | | LYAVGGHDGSSYLNSVERYDPKTNQWSSDVAPTSTCRTSVGVAVLGGFLYAVGGQDGVSCL |
| | | NIVERYDPKENKWTRVASMSTRRLGVAVAVLGGFLYAVGGSDGTSPLNTVERYNPQENRWH |
| | | TIAPMGTRRKHLGCAVYQDMIYAVGGRDDTTELSSAERYNPRTNQWSPVVAMTSRRSGVGL |
| | | AVVNGQLMAVGGEDGTTYLKTIEVFDPDANTWRLYGGMNYRRLGGGVGVIKMTHCESHIW |
| NP_059111.2 | >gi\|166235129\|ref\| | MEGESVKLSSQTLIQAGDDEKNQRTITVNPAHMGKAFKVMNELRSKQLLCDVMIVAEDVEI |
| (KLHL3) | NP_059111.2\|kelch-like 3 | EAHRVVLAACSPYFCAMFTGDMSESKAKKIEIKDVDGQTLSKLIDYITTAEIEVTEENVQV |
| SEQ ID NO: 14 | [Homo sapiens] | LLPAASLLQLMDVRQNCCDFLQSQLHPTNCLGIRAFADVHTCTDLLQQANAYAEQHFPEVM |
| | | LGEEFLSLSLDQVCSLISSDKLTVSSEEKVFEAVISWINYEKETRLEHMAKLMEHVRLPLL |
| | | PRDYLVQTVEEEALIKNNNTCKDFLIEAMKTHLLPLDQRLLIKNPRTKPRTPVSLPKVMIV |
| | | VGGQAPKAIRSVECYDFEEDRWDQTAELPSRRCRAGVVFMAGHVYAVGGENGSLRVRTVDV |
| | | YDGVKDQWTSIASMQERRSTLGAAVLNDLLYAVGGEDGSTGLASVEAYSYKTNEWFFVAPM |
| | | NTRRSSVGVGVVEGKLYAVGGYDGASRQCLSTVEQYNPATNEWIYVADMSTRRSGAGVGVL |
| | | SGQLYATGGHDGPLVRKSVEVYDPGTNTWKQVADMNMCRRNAGVCAVNGLLYVVGGDDGSC |
| | | NLASVEYYNPVTDKWTLLPTNMSTGRSTAGVAVIHKSL |
| NP_938073.1 | >gi\|38194229\|ref\| | MQPRSERPAGRTQSPEHGSPGPGPEAPPPPPPQPPAPEAERTRPRQARPAAPMEGAVQLLS |
| (KLHL17) | NP_938073.1\|kelch-like 17 | REGHSVAHNSKRHYHDAFVAMSRMRQRGLLCDIVLHVAAKEIRAHKVVLASCSPYFHAMFT |
| SEQ ID NO: 15 | [Homo sapiens] | NEMSESRQTHVTLHDIDPQALDQLVQFAYTAEIVVGEGNVQTLLPAASLLQLNGVRDACCK |
| | | FLLSQLDPSNCLGIRGFADAHSCSDLLKAAHRYVLQHFVDVAKTEEFMLLPLKQVLELVSS |
| | | DSLNVPSEEEVYRAVLSWVKHDVDARRQHVPRLMKCVRLPLLSRDFLLGHVDAESLVRHHP |
| | | DCKDLLIEALKFHLLPEQRGVLGTSRTRPRRCEGAGPVLFAVGGGSLFAIHGDCEAYDTRT |
| | | DRWHVVASMSTRRARVGVAAVGNRLYAVGGYDGTSDLATVESYDPVTNTWQPEVSMGTRRS |
| | | CLGVAALHGLLYSAGGYDGASCLNSAERYDPLTGTWTSVAAMSTRRRYVRVATLDGNLYAV |
| | | GGYDSSSHLATVEKYEPQVNVWSPVASMLSRRSSAGVAVLEGALYVAGGNDGTSCLNSVER |
| | | YSPKAGAWESVAPMNIRRSTHDLVAMDGWLYAVGGNDGSSSLNSIEKYNPRTNKWVAASCM |
| | | FTRRSSVGVAVLELLNFPPPSSPTLSVSSTSL |
| NP_001154993.1 | >gi\|239835722\|ref\| | MVWLEARPQILFVCTKQGHQKPLDSKDDNTEKHCPVTVNPWHMKKAFKVMNELRSQNLLCD |
| (KLHL2 | NP_001154993.1\| | VTIVAEDMEISAHRVVLAACSPYFHAMFTGEMSESRAKRVRIKEVDGWTLRMLIDYVYTAE |
| isoform 2) | kelch-like 2, Mayven | IQVTEENVQVLLPAAGLLQLQDVKKTCCEFLESQLHPVNCLGIRAFADMHACTDLLNKANT |
| SEQ ID NO: 16 | isoform 2 [Homo sapiens] | YAEQHFADVVLSEEFLNLGIEQVCSLISSDKLTISSEEKVFEAVIAWVNHDKDVRQEFMAR |
| | | LMEHVRLPLLPREYLVQRVEEEALVKNSSACKDYLIEAMKYHLLPTEQRILMKSVRTRLRT |
| | | PMNLPKLMVVVGGQAPKAIRSVECYDFKEERWHQVAELPSRRCRAGMVYMAGLVFAVGGFN |
| | | GSLRVRTVDSYDPVKDQWTSVANMRDRRSTLGAAVLNGLLYAVGGFDGSTGLSSVEAYNIK |
| | | SNEWFHVAPMNTRRSSVGVGVVGGLLYAVGGYDGASRQCLSTVECYNATTNEWTYIAEMST |
| | | RRSGAGVGVLNNLLYAVGGHDGPLVRKSVEVYDPTTNAWRQVADMNMCRRNAGVCAVNGLL |
| | | YVVGGDDGSCNLASVEYYNPTTDKWTVVSSCMSTGRSYAGVTVIDKPL |
| NP_009177.3 | >gi\|239835720\|ref\| | METPPLPPACTKQGHQKPLDSKDDNTEKHCPVTVNPWHMKKAFKVMNELRSQNLLCDVTIV |
| (KLHL2 | NP_009177.3\|kelch-like 2, | AEDMEISAHRVVLAACSPYFHAMFTGEMSESRAKRVRIKEVDGWTLRMLIDYVYTAEIQVT |
| isoform 1) | Mayven isoform 1 | EENVQVLLPAAGLLQLQDVKKTCCEFLESQLHPVNCLGIRAFADMHACTDLLNKANTYAEQ |
| SEQ ID NO: 17 | [Homo sapiens] | HFADVVLSEEFLNLGIEQVCSLISSDKLTISSEEKVFEAVIAWVNHDKDVRQEFMARLMEH |
| | | VRLPLLPREYLVQRVEEEALVKNSSACKDYLIEAMKYHLLPTEQRILMKSVRTRLRTPMNL |
| | | PKLMVVVGGQAPKAIRSVECYDFKEERWHQVAELPSRRCRAGMVYMAGLVFAVGGFNGSLR |
| | | VRTVDSYDPVKDQWTSVANMRDRRSTLGAAVLNGLLYAVGGFDGSTGLSSVEAYNIKSNEW |
| | | FHVAPMNTRRSSVGVGVVGGLLYAVGGYDGASRQCLSTVECYNATTNEWTYIAEMSTRRSG |
| | | AGVGVLNNLLYAVGGHDGPLVRKSVEVYDPTTNAWRQVADMNMCRRNAGVCAVNGLLYVVG |
| | | GDDGSCNLASVEYYNPTTDKWTVVSSCMSTGRSYAGVTVIDKPL |
| NP_079286.2 | >gi\|55925604\|ref\| | MVEDGAEELEDLVHFSVSELPSRGYGVMEEIRRQGKLCDVTLKIGDHKFSAHRIVLAASIP |
| (KLHL18) | NP_079286.2\|kelch-like 18 | YFHAMFTNDMMECKQDEIVMQGMDPSALEALINFAYNGNLAIDQQNVQSLLMGASFLQLQS |
| SEQ ID NO: 18 | [Homo sapiens] | IKDACCTFLRERLHPKNCLGVRQFAETMMCAVLYDAANSFIHQHFVEVSMSEEFLALPLED |
| | | VLELVSRDELNVKSEEQVFEAALAWVRYDREQRGPYLPELLSNIRLPLCRPQFLSDRVQQD |
| | | DLVRCCHKCRDLVDEAKDYHLMPERRPHLPAFRTRPRCCTSIAGLIYAVGGLNSAGDSLNV |
| | | VEVFDPIANCWERCRPMTTARSRVGVAVVNGLLYAIGGYDGQLRLSTVEAYNPETDTWTRV |
| | | GSMNSKRSAMGTVVLDGQIYVCGGYDGNSSLSSVETYSPETDKWTVVTSMSSNRSAAGVTV |
| | | FEGRIYVSGGHDGLQIFSSVEHYNHHTATWHPAAGMLNKRCRHGAASLGSKMFVCGGYDGS |
| | | GFLSIAEMYSSVADQWCLIVPMHTRRSRVSLVASCGRLYAVGGYDGQSNLSSVEMYDPETD |
| | | CWTFMAPMACHEGGVGVGCIPLLTI |

We claim:

1. A method of diagnosing primary biliary cirrhosis (PBC) in an individual comprising:
a. contacting a test sample from the individual with one or more target antigens of kelch-like 12, a homolog of kelch-like 12, or a fragment thereof, said fragment comprising an epitope of said antigen; and
b. detecting binding of said one or more target antigens or said fragments thereof comprising an epitope to one or more antibodies in the test sample; and
c. comparing the level of autoantibody to a control sample from a healthy individual;
wherein an increased level of the one or more antibodies bound to the one or more target antigens or fragments thereof as compared to said control sample from said healthy individual is indicative of primary biliary cirrhosis (PBC).

2. The method of claim 1, wherein the one or more target antigens or fragments thereof comprising an epitope are immobilized on a solid support.

3. The method of claim 1, wherein said homolog of kelch-like 12 comprises a sequence selected from the group consisting of SEQ ID NOS: 12-18.

4. The method of claim 1, wherein the test sample is contacted with two or more of the target antigens or fragments thereof comprising an epitope.

5. The method of claim 1, wherein the test sample is contacted with three or more of the target antigens or fragments thereof comprising an epitope.

6. The method of claim 1, wherein the test sample is contacted with four or more of the target antigens or fragments thereof comprising an epitope.

7. The method of claim 1, wherein the test sample is contacted with five or more of the target antigens or fragments thereof comprising an epitope.

8. The method of claim 1, wherein the test sample is contacted with six or more of the target antigens or fragments thereof comprising an epitope.

9. The method of claim 1, wherein the test sample is cells, tissues or body fluids.

10. The method of claim 1, wherein the test sample is blood, plasma or serum.

11. The method of claim 1, wherein the one or more purified recombinant target antigens or fragments thereof comprising an epitope are produced recombinantly.

12. The method of claim 11, wherein the one or more recombinant target antigens or fragments thereof comprising an epitope are produced using cell-free protein expression.

13. The method of claim 1, wherein the one or more purified recombinant target antigens or fragments thereof comprising an epitope further comprise a tag sequence located at the C-terminal or N-terminal, or at both the C-terminal and N-terminal.

14. The method of claim 1, wherein the detection step further comprises a labeled anti-immunoglobulin (IG) antibody and wherein the one or more target antigen or fragments thereof, autoantibody, and anti-IG antibody form a complex.
